(19) United States
(12) Patent Application Publication
(10) Pub. No.: US 2020/0181670 A1
(43) Pub. Date: Jun. 11, 2020

(54) Improved In Vitro Transcription/Translation (TXTL) System and Use Thereof

(71) Applicant: Synvitrobio, Inc., San Francisco, CA (US)

(72) Inventors: Zachary Z. Sun, San Francisco, CA (US); Abel C. Chiao, San Francisco, CA (US); Dan E. Robertson, San Francisco, CA (US); Louis E. Metzger, IV, San Francisco, CA (US); Richard Mansfield, San Francisco, CA (US); Kelly S. Trego, San Francisco, CA (US)

(21) Appl. No.: 16/638,272

(22) PCT Filed: Aug. 13, 2018
(86) PCT No.: PCT/US2018/046477

§ 371 Date: Feb. 11, 2020

Related U.S. Application Data
(60) Provisional application No. 62/544,228, filed on Aug. 11, 2017.

(51) Int. Cl. C12P 21/02; C12N 15/85

(52) U.S. Cl. CPC C12P 21/02; C12Y 207/07006; C12N 15/85

(57) ABSTRACT

Provided herein, in one aspect, is a composition for in vitro transcription and translation, comprising: a treated cell lysate derived from one or more host cells such as bacteria, archaea, plant or animal; a plurality of supplements for gene transcription and translation; an energy recycling system for providing and recycling adenosine triphosphate (ATP); and one or more exogenous additives. Methods for making and using the same are also provided.

### CROSS-REFERENCE TO RELATED APPLICATIONS

This application claims priority to and the benefit of U.S. Provisional Application No. 62/544,228 filed Aug. 11, 2017, the entire disclosure of all of which is hereby incorporated by reference.

### STATEMENT OF GOVERNMENT INTEREST

This invention was made with government support under contract number W911NF17C0008 awarded by the U.S. Defense Advanced Research Projects Agency (DARPA), and grant number 1R43AT00952201 awarded by the U.S. National Institutes of Health (NIH). The government has certain rights in the invention.

### FIELD

The disclosure relates to cell-free compositions and use thereof, particularly improved compositions for conducting cell-free (in vitro) transcription and translation.

### BACKGROUND

Synthetic biology has emerged as a useful approach to decoding fundamental laws underlying biological control. Recent efforts have produced many systems and approaches and generated substantial insights on how to engineer biological functions and efficiently optimize synthetic pathways.

Despite efforts and progresses, current approaches to perform such engineering are often laborious, costly and difficult. Challenges still remain in developing engineering-driven approaches and systems to accelerate the design-build-test cycles required for reprogramming existing biological systems, constructing new biological systems and testing genetic circuits for transformative future applications in diverse areas including biology, engineering, green chemistry, agriculture and medicine.

An in vitro transcription-translation cell-free system (Sun et al., 2013) has been developed which allows for the rapid prototyping of genetic constructs in an environment that behaves similarly to a cell (Niederholtmeyer, Sun, Hon, & Yeung, 2015). One of the main purposes of working in vitro is to be able to generate fast speeds—in vitro, reactions can take 8 hours and can scale to thousands of reactions a day, a multi-fold improvement over similar reactions in cells. Despite the potential of this cell-free system, it needs be fine-tuned when used in different applications to achieve optimal results.

A need therefore exists for improved cell-free systems, particularly systems with improved transcription and translation efficiency.

### SUMMARY

Disclosed herein are improved in vitro transcription/translation (TXTL) systems and use thereof.

In one aspect, a composition for in vitro gene expression is provided, comprising: a treated cell lysate derived from one or more host cells such as bacteria, archaea, plant or animal; a plurality of supplements for gene transcription and translation; an energy recycling system for providing and recycling adenosine triphosphate (ATP); and one or more exogenous additives selected from the group consisting of polar aprotic solvents, quaternary ammonium salts, betaines, sulfones, ectoines, glycols, amides, amines, sugar polymers, sugar alcohols, slow elongation-rate RNA polymerase (RNAP) and ribosomes, wherein the sugar polymers and sugar alcohols are not for providing energy source.

The composition can be used in expressing a metagenomically derived gene, a plurality of genes that together constitute a pathway, and/or synthetic proteins, wherein preferably the pathway is designed for synthesis of a natural product. In some embodiments, the gene or pathway has not been optimized for in vitro gene expression.

In some embodiments, the plurality of supplements can include magnesium and potassium salts, ribonucleotides, amino acids, a starting energy substrate, and a pH buffer.

In certain embodiments, the one or more additives can modulate nucleic acid secondary structure, improve RNAP processivity and/or stability, affect RNAP elongation rate, improve ribosome synergy with RNAP and/or stability, and/or improve stability of polypeptide being synthesized.

In some embodiments, the slow elongation-rate RNAP can be homologous to the host cells, such as RNA PolI, RNA PolII, RNA PolIII, and bacterial RNAP. In some embodiments, the slow elongation-rate RNAP can be heterologous to the host cells, such as SP6 RNAP variants, T7 RNAP variants, and T3 RNAP variants. In some embodiments, the slow elongation-rate RNAP can be sourced from a thermophile or psychrophile. In some embodiments, the slow elongation-rate RNAP can be a synthetic RNAP such as engineered T7 RNAP variants and engineered RNA PolII variants. In some embodiments, the slow elongation-rate RNAP can be engineered by directed evolution and/or rational design. In some embodiments, the slow elongation-rate RNAP can be provided as a purified protein or as a nucleic acid encoding the slow elongation-rate RNAP.

The composition can, in some embodiments, further include exogenous nucleic acids to be expressed in the composition, wherein each exogenous nucleic acid comprises a promoter that is recognized by the slow elongation-rate RNAP.

In some embodiments, the ribosomes can be sourced from the host cells, or from an organism different than the host cells, wherein preferably the ribosomes are provided at 0.1 μM to 100 μM concentration.

In some embodiments, the composition can include both slow elongation-rate RNAP and exogenous ribosomes, wherein preferably the slow elongation-rate RNAP and the exogenous ribosomes are coupled, wherein optionally such coupling is orthogonal to the host cells.

In another aspect, a method of preparing the composition disclosed herein is provided, comprising: providing an in vitro transcription/translation system comprising the treated cell lysate, the plurality of supplements and the energy recycling system; and supplying the one or more exogenous additives disclosed herein.

In a further aspect, a method of in vitro gene expression is provided, comprising: providing the composition disclosed herein, and providing one or more nucleic acids to be expressed.

### BRIEF DESCRIPTION OF THE DRAWINGS

FIG. 1 provides an overview of cell-free expression. In cell-free expression, a host is converted into a lysate and supplied with factors to enable the conversion of DNA to mRNA and protein.

FIG. 2 provides a comparison of traditional heterologous expression to cell-free expression.

FIG. 3 shows the effect of dimethyl sulfoxide (DMSO) on transcription of a non-model gene from 16 nM linear DNA of sigma70-lazC, in the presence of 0-10% DMSO (working concentration), by Malachite green (Mg)-aptamer (left). The right figure shows protein yield measured by SDS-PAGE tracking FloroTect™ incorporation.

FIG. 4 shows TXTL expression of 6 nM linear DNA of sigma70-mcjC, in the presence of 4% DMSO, 800 mM betaine, 400 mM betaine, and nothing (neg. ctrl.). The black arrow represents the expected size of mcjC. Other bands on the gel can be used to normalize protein expression levels.

FIG. 5 shows TXTL expression of 6 nM linear DNA of multiple genes (MBP, klebB, klebC, mcjB, and mcjC) from sigma70 (and sigma70(lacO1)) promoters, with varying concentrations of betaine. The black arrows represent the expected size of each protein. Other bands on the gel can be used to normalize protein expression levels.

FIG. 6 shows expression of two proteins, a MBP variant and GFP, in Streptomyces coelicolor TXTL. Left, right three lanes, an SDS-PAGE gel tracking production of MBP variant from 15 nM of linear DNA in S. coelicolor TXTL with 1% DMSO, no additives (neg. ctrl.), or 400 mM betaine. Arrow, expected size of MBP variant. The left lane is a sample E. coli TXTL expressing a different MBP variant. Right, 6 nM of linear DNA GFP expression in S. coelicolor TXTL after 12 hours with varying concentrations of betaine. afu, arbitrary fluorescence units.

FIG. 7 plots the TXTL expression of multiple T7 RNA polymerase (RNAP) promoter variants expressing GFP from either 16 nM linear or 8 nM plasmid DNA, with expression of a negative control also plotted. Error bars represent 1 standard deviation from 2 experiments.

FIG. 8 plots the Mg-aptamer expression of a metagenomic coding sequence from 8 nM plasmid DNA or 16 nM linear DNA, driven either from a sigma70 promoter or a T7 promoter. Right, an SDS-PAGE gel tracking FloroTect™ showing resulting protein from each reaction produced on a gel, where the black arrow indicates the expected protein.

FIG. 9 shows a SDS-PAGE gel tracking FloroTect™ of the TXTL expression of non-model genes klebB and klebC from 2-8nM of sigma70 linear DNA and from 4 nM of T7 promoters linear DNA, where for the T7 promoters different T7 RNAP variants are co-expressed (wildtype, Q649S, G645A, I810S) from 1-1.5 nM of linear DNA. WT, wildtype, QS, Q649S, GA, G645A, IS, I810S. White arrows represent RNAP expected size; black arrows represent klebB and klebC expected protein size.

FIG. 10 shows kinetic data tracking the binding of FlAsH-EDT2 to a tagged MBP in TXTL. Left, controls showing 4 nM of linear DNA expressing MBP-“CCPGCC” (all non “/c”) or MBP without tag (“/c”) co-expressed with 1 nM-4 nM of different linear DNA expressed T7 RNAP variants (WT, Q649S, G645A, I810S). Right, expression of 4 nM of linear DNA expressing MBP-“CCPGCC” with 4 nM of different linear DNA expressed T7 RNAP variants.

FIG. 11 shows the peak translation rate of E. coli TXTL reactions of 8 nM sigma70-GFP, where S70 ribosomes are supplemented from 0-2 μM working concentration and magnesium is supplemented from 0-2 mM. afu, arbitrary fluorescence units.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The improved in vitro transcription/translation (TXTL) system disclosed herein can more efficiently catalyze information flow from DNA to cellular function. It improves upon prior systems by broadening its utility for bioengineering and biodiscovery. In some embodiments, the systems and compositions disclosed herein are designed to promote synergies between the transcription and translation process components of its derivative organism. The compositional modifications can be implemented for an in vitro system derived from any organism. In certain embodiments, the system can include an isolated gene expression machinery of a derivative organism, which can be free of the burden of in vivo metabolism, cell regulation systems, and endogenous DNA expression. Such system can be used for rapidly observing gene expression, gene product assembly and function. By virtue of its ability to accelerate gene expression, the systems and compositions disclosed herein overcome previously limiting barriers of heterologous expression, producer organisms' unculturability and the variability in coupling efficiency of in vitro expression.

For example, when applied to bioengineering, the compositions and methods disclosed herein can enable high-throughput expression and activity prototyping, accelerating design/build/test cycles for synthetic biology, metabolic engineering, bioprocess development, or convergent cycles of gene, pathway and genetic element evolution. When used for biodiscovery, the compositions and methods disclosed herein can remove largely unsolved barriers to conventional gene expression in heterologous hosts, opening vast areas of gene sequence space for exploration; via expression of genes from uncultured organisms, microbiomes, libraries of cryptic genes and clusters.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

As used herein, the terms “nucleic acid,” “nucleic acid molecule” and “polynucleotide” may be used interchangeably and include both single-stranded (ss) and double-stranded (ds) RNA, DNA and RNA:DNA hybrids. These terms are intended to include, but are not limited to, a polymeric form of nucleotides that may have various lengths, including deoxyribonucleotides and/or ribonucleotides, or analogs or modifications thereof. A nucleic acid molecule may encode a full-length polypeptide or RNA or a fragment of any length thereof, or may be non-coding.

As used herein, the terms “gene” and “coding sequence” may be used interchangeably and refer to a sequence of polynucleotides, the order of which determines the order of amino acid monomers in a polypeptide or RNA molecule which a cell (or virus) may synthesize.

Nucleic acids can be naturally-occurring or synthetic polymeric forms of nucleotides. The nucleic acid molecules of the present disclosure may be formed from naturally-occurring nucleotides, for example forming deoxyribonucleic acid (DNA) or ribonucleic acid (RNA) molecules. Alternatively, the naturally-occurring oligonucleotides may include structural modifications to alter their properties, such as in peptide nucleic acids (PNA) or in locked nucleic acids (LNA). The terms should be understood to include equivalents, analogs of either RNA or DNA made from nucleotide analogs and as applicable to the embodiment being described, single-stranded or double-stranded polynucleotides. Nucleotides useful in the disclosure include, for example, naturally-occurring nucleotides (for example, ribonucleotides or deoxyribonucleotides), or natural or synthetic modifications of nucleotides, or artificial bases. Modifications can also include phosphorothioated bases for increased stability.

As used herein, unless otherwise stated, the term “transcription” refers to the synthesis of RNA from a DNA template; the term “translation” refers to the synthesis of a polypeptide from an mRNA template. Translation in general is regulated by the sequence and structure of the 5′ untranslated region (5′-UTR) of the mRNA transcript. One regulatory sequence is the ribosome binding site (RBS), which promotes efficient and accurate translation of mRNA. The prokaryotic RBS is the Shine-Dalgarno sequence, a purine-rich sequence of 5′-UTR that is complementary to the UCCU core sequence of the 3′-end of 16S rRNA (located within the 30S small ribosomal subunit). Various Shine-Dalgarno sequences have been found in prokaryotic mRNAs and generally lie about 10 nucleotides upstream from the AUG start codon. Activity of a RBS can be influenced by the length and nucleotide composition of the spacer separating the RBS and the initiator AUG. n eukaryotes, the Kozak sequence lies within a short 5′ untranslated region and directs translation of mRNA. An mRNA lacking the Kozak consensus sequence may also be translated efficiently in an in vitro system if it possesses a moderately long 5′-UTR that lacks stable secondary structure. While E. coli ribosome preferentially recognizes the Shine-Dalgarno sequence, eukaryotic ribosomes (such as those found in retic lysate) can efficiently use either the Shine-Dalgarno or the Kozak ribosomal binding sites.

As used herein, the term “coupling” or “coupled” refers to the concerted action of the DNA transcription and mRNA translation systems as well as the innate folding factors in the lysate promoting protein folding, where fidelity, kinetics and cooperativity determine productivity of active protein. Degree of coupling is a measure of the efficiency of information translation and amplification into functional protein and is equivalent to the extent of amplification of gene copy to active protein. In some embodiments, efficient coupling minimizes the formation of untranslated mRNA, truncated mRNA, mRNA secondary structure, and/or degradation by endonucleases and/or exonuclease. In various embodiments, efficient coupling optimizes full-length transcript synthesis, lifetime of mRNA transcript, ribosome translation elongation-rate and/or protein folding efficiency.

As used herein, the term “host” or “host cell” refers to any prokaryotic or eukaryotic single cell (e.g., yeast, bacterial, archaeal, etc.) or organism. The host cell can be a recipient of a replicable expression vector, cloning vector or any heterologous nucleic acid molecule. Host cells may be prokaryotic cells such as species of the genus Escherichia or Lactobacillus, or eukaryotic organisms such as yeast or tobacco. The heterologous nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Green & Sambrook, 2012, Molecular Cloning: A laboratory manual, 4th ed., Cold Spring Harbor Laboratory Press, New York, incorporated herein by reference.

As used herein, an item that is “homologous” or “native” (used interchangeably) to a host organism, such as an enzyme, polymerase, gene, or protein, is one that originates from the host. and is the same as the original item in the host or exists as non-engineered or engineered variant of the host. This contrasts with “heterologous” or “non-native,” which is not naturally found in the host organism and instead originates from a different organism or species, which can exist in its original form or as a non-engineered or engineered variant.

As used herein, the term “orthogonal” refers to a system whose basic structure or the way in which components within the system interact with one another is so dissimilar to those occurring in nature, or to those to which the system is being compared, such that interaction between the system and either nature or the system being compared is limited (if any).

As used herein, the term “sigma70” refers to a promoter is recognized by a housekeeping sigma factor in a native host and/or a TXTL system made from the native host. In various embodiments, it may be specifically the OR2OR1Pr promoter present on construct #40019, Addgene, or may be a pLacO1 promoter or variant (Lutz & Bujard, 1997). The preparation of genetic material incorporating this promoter can be found in Green & Sambrook, 2012, Molecular Cloning: A laboratory manual, 4th ed, Cold Spring Harbor Laboratory Press, New York, incorporated herein by reference, and other laboratory manuals.

The term “engineer,” “engineering” or “engineered,” as used herein, refers to genetic manipulation or modification of biomolecules such as DNA, RNA and/or protein, or like technique commonly known in the biotechnology art.

The term “variant” or “variant form” in the context of a polypeptide refers to a polypeptide that is capable of having at least 10% of one or more activities of the naturally-occurring sequence. In some embodiments, the variant has substantial amino acid sequence identity to the naturally-occurring sequence, or is encoded by a substantially identical nucleotide sequence, such that the variant has one or more activities of the naturally-occurring sequence. In the context of a chemical, “variant” refers to a derivative that can be viewed to arise or actually be synthesized from a parent chemical by replacement of one or more atoms with one or more substituents. Common substituents include, e.g., alkyl, haloalkyl, cycloalkyl, heterocyclyl, heterocycloalkenyl, cycloalkenyl, aryl, or heteroaryl groups.

As described herein, “genetic module” and “genetic element” may be used interchangeably and refer to any coding and/or non-coding nucleic acid sequence. Genetic modules may be operons, genes, gene fragments, promoters, exons, introns, regulatory sequences, tags, or any combination thereof. In some embodiments, a genetic module refers to one or more of coding sequence, promoter, terminator, untranslated region, ribosome binding site, polyadenlylation tail, leader, signal sequence, vector and any combination of the foregoing. In certain embodiments, a genetic module can be a transcription unit as defined herein.

As used herein, “metagenomic” or “metagenome” means genetic material originating from an environmental sample. The genetic material is typically, but does not have to be exclusively, from microbes. Metagenomic material is typically “non-model” as well, in that it has not been optimized to express well in a heterologous and/or cell-free system.

As used herein, “thermophile” refers to a microorganism with optimal growth at a temperature of 40 Celsius or higher. Examples include species from Pyrococcus, Pyroglobus, Thermococcus, without limitation.

As used herein, “psychrophile” refers to a microorganism with optimal growth at a temperature of 15 Celsius or lower. Examples include species from Arthrobacter, Psychrobacter, Synechococcus, without limitation.

The term “additive” refers to an addition, whether chemical or biological in nature, whether natural or synthetic, that is provided to a system. In some embodiments, the additive disclosed herein is provided exogenously, e.g., from an external source.

As used herein, “polar aprotic solvents” are compounds which are liquid at room temperature, which lack a hydrogen-bond donor atom, which possess dielectric constants >6, which possess dipole moments >1, and which contain at least one potential hydrogen-bond acceptor atom. In some embodiments, additions include polar aprotic solvents, diethylsulfoxide, acetonitrile, acetone, N-methyl-2-pyrrolidone, tetrahydrofuran, and/or propylene carbonate, without limitation. In some embodiments, the polar aprotic solvents can be provided at concentration ranges of about 0.1-10% vol/vol. In some embodiments, the polar aprotic solvents can be added as individual chemicals to the cell-free reaction. In some embodiments, dimethyl sulfoxide is excluded from the polar aprotic solvents as disclosed herein. In some embodiments, acetate is excluded from the polar aprotic solvents as disclosed herein, when added to a cell-free reaction as a salt form (e.g., Magnesium acetate, Potassium acetate).

As used herein, “quaternary ammonium salts” are salts containing an ammonium cation. This cation contains a nitrogen possessing a permanent positive charge, which is bonded to four chemical substituents. These substituents may be the same as each other, or singly, doubly, triply, or completely different from each other. In some embodiments, the quaternary ammonium salts include benzalkonium chloride, tetramethylammonium chloride, and/or tetrabutylammonium phosphate, without limitation. In some embodiments, the quaternary ammonium salts can be provided at concentration ranges of about 0.001-1.5 M. In some embodiments, betaine, trimethylglycine, and/or variants of betaine are included. In some embodiments, betaine, trimethylglycine, and/or variants of betaine are provided at concentration ranges of about 0.1 M-1.5 M, more preferably at concentration ranges of about 200 mM-600 mM, about 300-500 mM, or about 400 mM. In some embodiments, betaine, trimethylglycine, and/or variants of betaine are not for stabilizing nucleic acid products, but rather for serving as crowding reagents and otherwise promoting TXTL product stability. In some embodiments, caldohexamine, tetrakis(3-aminopropyl) ammonium, and/or tris(3-aminopropyl)amine are excluded from the quaternary ammonium salts or betaines disclosed herein.

As used herein, “sulfones” are compounds containing a hexavalent sulfur atom that is doubly bonded to two oxygens, and is singly bonded to two additional substituents which are usually, but not always, carbons. In some embodiments, the sulfones include propylsulfoxide, n-butylsulfoxide, methyl sulfone, methyl butyl sulfoxide, sulfolane, tetramethylene sulfoxide, and/or ethyl sulfone, without limitation. In some embodiments, the sulfones can be provided at concentration ranges of about 0.01 M-1.5 M.

As used herein, “ectoines” are 1,4,5,6-tetrahydro-2-methyl-4-pyrimidinecarboxylic acid and derivatives thereof. Ectoines can be naturally produced by microorganisms as osmolytes for protection against osmotic stress. In some embodiments, the ectoines can include L-ectoine, alpha-hyroxyectoine, and/or homoectoine, without limitation. In some embodiments, the sulfones can be provided at concentration ranges of about 0.01 M-1.5 M.

As used herein, “glycols” are compounds that have two hydroxyl groups, separated from each other by some number of atoms greater than or equal to two. In some embodiments, the glycols can include glycerol, ethylene glycol, and/or neopentyl glycol, without limitation. In some embodiments, the glycols can include polyethylene glycols, e.g., at concentrations greater than about 0.1% w/vol but less than about 30% w/vol and at sizes greater than about 10,000 dalton in molecular weight. In some embodiments, the glycols can include polyethylene oxide at concentrations greater at concentrations greater than about 0.1% w/vol but less than about 30% w/vol.

As used herein, “amides” are compounds having the formula compound with the functional group RₙE(O)ₓNR′₂, where R and R′ are either hydrogen or common substituents (e.g., alkyl, alkenyl, etc.) attached via non-hydrogen atoms. As used herein, the amines can be compounds which contain a lone pair of electrons on a basic nitrogen atom. In some embodiments, amides and amines include formamide, acetamide, 2-pyrrolidone, propionamide, N-methyl formadine, N,N-dimethyl formadine, formyl pyrrolidine, formyl piperdine, and/or formyl morpholine, without limitation. In some embodiments, amines and amides can be provided at concentration ranges of about 0.001 M-0.05 M. In some embodiments, spermidine, spermine, thermospermine, caldopentamine, homospermine, homocaldopentamine, putrescine, and/or tetraamine are excluded.

As used herein, “sugar polymers” are linked versions with identical or dissimilar sugars (oligosaccharides, such as maltodextrin, α-cyclodextrin, etc.). As used herein, “sugar alcohols”, which are usually derived from sugars, are polyols. Polyols are hydrocarbons that contain more than two hydroxyl groups. In some embodiments, the sugar polymers and sugar alcohols disclosed herein are not used for an energy source and/or are not metabolized by the cell-free reaction. In some embodiments, the sugar polymers can include alpha-cyclodextrin and/or trehalose, without limitation. In some embodiments, the sugar alcohols can include xylitol, D-threitol, and/or sorbitol, without limitation. In some embodiments, the sugar polymers can exclude maltodextrin, glycogen, and maltose.

As used herein, a “slow elongation-rate” polymerase is a polymerase that has an in vitro elongation rate between about 10 and 120 nucleotides per second (nt/s), more preferably between about 10 and 50 nt/s. This polymerase is designed to be as close as possible to the elongation rate of a native polymerase from the original host. In various embodiments, “elongation-rate” is also referred to as “speed.” Elongation rate can be measured as described in (Bonner, Lafer, & Sousa, 1994) and in (Golomb & Chamberlin, 1974), incorporated by reference, as a nucleotide per second rate.

As used herein, “processivity” of a polymerase refers to the polymerase's ability to catalyze consecutive reactions without releasing its substrate. Processivity can be measured as described in (Bonner et al., 1994) and in (McClure & Chow, 1980), incorporated by reference, typically as a fraction from about 0.70 to 1. A “high processivity” polymerase refers to one that is between about 0.80 to 0.99, or between about 0.90 to 0.99.

As used herein, “rational design” is the process of making mutations in a gene in order to vary the function of the resulting enzyme. This process is typically informed by physical models of activity, where motifs that effect desired activity are known. This process is demonstrated for a model polymerase in (Sousa, Chung, Rose, & Wang, 1993) and incorporated by reference.

As used herein, “directed evolution” is the process of using evolutionary pressure and mimicking natural selection to evolve an enzyme to perform a desired function. This process involves producing significant amounts of genetic variation. Examples of directed evolution methods included phage-assisted continuous evolution by (Esvelt, Carlson, & Liu, 2011), and other methods detailed in (Renata, Wang, & Arnold, 2015), incorporated by reference.

Other terms used in the fields of recombinant nucleic acid technology and molecular and cell biology as used herein will be generally understood by one of ordinary skill in the applicable arts.

### Composition of In Vitro Transcription and Translation

The in vitro transcription and translation system is a system that is able to conduct transcription and translation outside of the context of a cell. In some embodiments, this system is also referred to as “cell-free system”, “cell-free transcription and translation”, “TX-TL”, “TXTL”, “lysate systems”, “in vitro system”, “ITT”, or “artificial cells.” In vitro transcription and translation systems can be either purified protein systems, that are not made from hosts, or can be made from a host strain that is formed as a “lysate.” Those skilled in the art will recognize that an in vitro transcription and translation requires transcription and translation to occur, and therefore does not encompass reactions with purified enzymes.

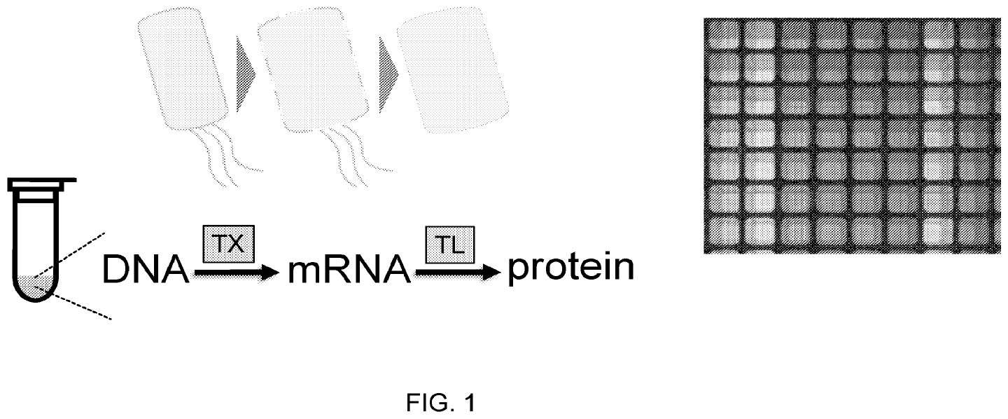

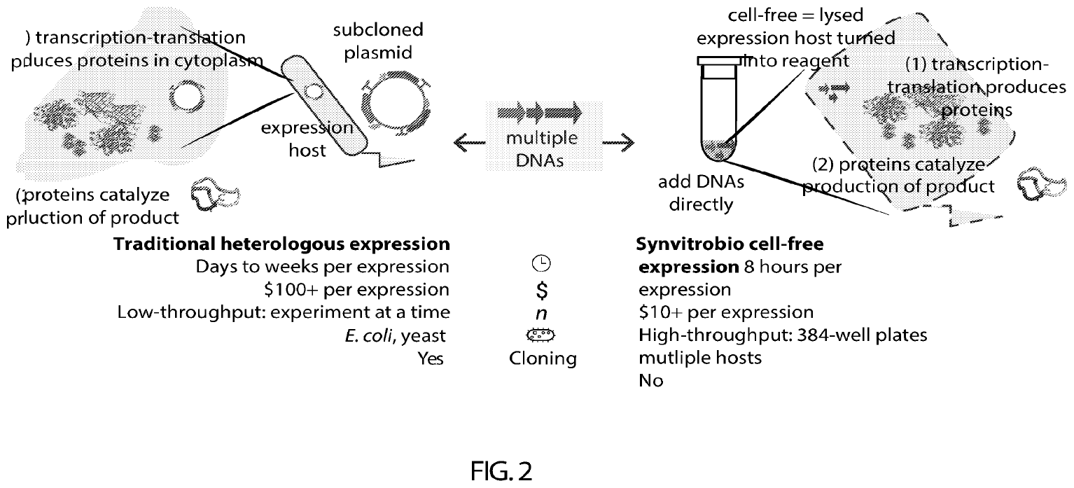

Cell-free transcription-translation is described in FIG. 1. Top, cell-free expression that takes in DNA and produces protein that catalyzes reactions. Bottom, diagram of cell-free production and representative data collected in 384-well plate format of GFP expression. Cell-free approaches contrasted to cellular approaches are described in FIG. 2. Cell-free platform allows for protein expression from multiple genes without live cells. Cell-free production biotechnology methods produce lysates from prokaryotic cells that are able to take recombinant DNA as input and conduct coupled transcription and translation to output enzymatically active protein. Cell-free systems take only 8 hours to express, rather than days to weeks in cells, since there is no need for cloning and transformation. They are also at least 10-fold cheaper to run than cells, and can be run in high-throughput as reactions are the equivalent of a reagent and used in a 384-well plate. Typical yields of prokaryotic systems are 750 μg/mL of GFP (30 μM). Extracts from multiple cell-free systems can be implemented, conducted at scales from 10 μl up to 10 mL.

Directions on how to make the lysate component of cell-free systems, particularly from E. coli, can be found in (Sun et al., 2013), which is incorporated by reference. While this procedure is adapted for E. coli cell-free systems, it can be used to produce other cell-free systems from other organisms and hosts (prokaryotic, eukaryotic, archaea, fungal, etc.) Examples, without limitation, of the production of other cell-free systems include Streptomyces spp. (Thompson, Rae, & Cundliffe, 1984), Bacillus spp. (Kelwick, Webb, MacDonald, & Freemont, 2016), and Tobacco BY2 (Buntru, Vogel, Spiegel, & Schillberg, 2014), where directions are incorporated by reference. The process for producing lysates in this disclosure involves growing a host in a rich media to mid-log phase, followed by washes, lysis by French Press and/or Bead Beating Homogenization, and clarification. A lysate that has been processed as such can be referred to as a “lysate”, a “treated cell lysate”, or an “extract”.

A plurality of supplements can be supplied along-side an extract to maintain gene expression. This includes necessary items for transcription and translation, such as amino acids, nucleotides (e.g., ribonucleotides), salts (Magnesium and Potassium), a source of energy, and a pH buffering component. A review of supplements can be found in (Chiao, Murray, & Sun, 2016), incorporated by reference. This can also include optional items that assist transcription and translation, such as cofactors, elongation factors, nanodiscs, vesicles, and antifoaming agents. These can also include additives to protect DNA, such as gamS, chi site-DNA, or other DNA protective agents.

An energy recycling system is necessary to drive synthesis of mRNA and proteins by providing ATP to a system and by maintaining system homoeostasis by recycling ADP to ATP, by maintaining pH, and generally supporting a system for transcription and translation. A review of energy recycling systems can be found in (Chiao et al., 2016), incorporated by reference. Examples, without limitation, of energy recycling systems that can be used include 3-PGA (Sun et al., 2013), PANOx (D.-M. Kim & Swartz, 2001), and Cytomim™ (Jewett & Swartz, 2004).

In some embodiments, a nucleic acid (e.g., DNA) can be supplied to produce a polypeptide from the nucleic acid by utilizing transcription and translation machinery in the in vitro TXTL system. The nucleic acid can include a gene or gene fragment as well as regulatory regions, such as promoter (e.g., OR2OR1Pr promoter, T7 promoter or T7-lacO promoter) and RBS region, such as the UTR1 from lambda phage, as described in (Shin & Noireaux, 2012). The nucleic acid can be linear or in the form of a plasmid.

In other embodiments, an mRNA can be supplied that utilizes translational components in the in vitro TXTL system to produce polypeptides. This mRNA can be from a purified natural source, or from a synthetically generated source, or can be generated in vitro, e.g., from an in-vitro transcription kit such as HiScribe™, MAXIscript™, MEGAscript™, mMESSAGE MACHINE™, MEGAshortscript™.

In some embodiments, the in vitro transcription and translation system can be used to express a metagenomically derived gene, a plurality of genes that together constitute one or more pathways (e.g., for synthesizing one or more natural products), and/or synthetic proteins. By using an in vitro TXTL system, the genes, pathways, or proteins can be rapidly expressed and diagnosed for their activity and function. To properly diagnose function, exogenous additives can be added to assist transcription, translation, coupling, and/or expression amounts. While certain model genes, pathways, or proteins that have been well studied may express well in TXTL systems, how to express non-model (less studied and less understood) genes, pathways, or proteins remain a critical issue requiring significant exploration. Many genes that are metagenomically-derived are non-model genes. Provided herein are additives that can generally and unexpectedly improve expression of various genes/pathways including non-model genes/pathways, which is significant and advantageous in improving in vitro TXTL of these genes/pathways and in turn, helping researchers understand these genes/pathways.

### Chemical Additives for Improving In Vitro Transcription and Translation

In some embodiments, chemical additives can be added to improve in vitro transcription and translation. Without wishing to be bound by theory, these additives are believed to act by reducing DNA template and mRNA secondary structures, to enhance the stability of the transcriptional machinery in the cell-free lysate, to enhance protein translation in the cell-free lysate by stabilizing/enhancing translational machinery, to promote folding of translated proteins, and/or to stabilize translated proteins, and/or to reduce proteolysis of translated proteins.

It is unexpected that certain exogenous additives can generally improve in vitro transcription and translation. This is especially true for non-model genes and/or metagenomically derived genes, where the gene is not optimized for transcription and translation. It has been surprisingly demonstrated herein that certain chemical additives can improve transcription and/or translation with previously unknown mechanisms of action. Exemplary additives are listed below.

| Additive Category | Compound Name | Concentration Range |
|---|---|---|
| Polar aprotic | diethylsulfoxide (DESO) | About 0.1-10% vol/vol |
| solvents | acetonitrile | About 0.1-10% vol/vol |
| | acetone | About 0.1-10% vol/vol |
| | N-methyl-2-pyrrolidone (NMP) | About 0.1-10% vol/vol |
| | tetrahydrofuran (THF) | About 0.1-10% vol/vol |
| | propylene carbonate | About 0.1-30% vol/vol |
| Quaternary | benzalkonium chloride | About 0.001-0.1 Molar (M) |
| ammonium salts | Tetramethylammonium chloride (TMAC) | About 0.001-0.1 Molar (M) |
| | Tetrabutylammonium phosphate (TBAP) | About 0.001-0.01 Molar (M) |
| | Betaine (trimethylglycine) | About 0.1-1.5 Molar (M) |
| Sulfones | propylsulfoxide | About 0.05--1.5 Molar (M) |
| | n-butylsulfoxide | About 0.05-0.5 Molar (M) |
| | methyl sulfone | About 0.5-1.5 Molar (M) |
| | methyl butyl sulfoxide | About 0.01-0.5 Molar (M) |
| | sulfolane | About 0.05-1 Molar (M) |
| | tetramethylene sulfoxide | About 0.01-1 Molar (M) |
| | ethyl sulfone | About 0.05-0.5 Molar (M) |
| Ectoines | L-ectoine | About 0.001-2 Molar (M) |
| | alpha-hyroxyectoine | About 0.001-2 Molar (M) |
| | homoectoine | About 0.001-2 Molar (M) |
| Glycols | polyethylene glycols (all sizes) | About 0.1-30% w/vol |
| | glycerol | About 0.001-2 Molar (M) |
| | ethylene glycol | About 0.001-4 Molar (M) |
| Amides and | formamide | About 0.001-0.5 Molar (M) |
| amines | acetamide | About 0.001-0.5 Molar (M) |
| | 2-pyrrolidone | About 0.001-0.5 Molar (M) |
| | propionamide | About 0.001-0.5 Molar (M) |
| | N-methyl formadine | About 0.001-0.5 Molar (M) |
| | N,N-dimethyl formadine | About 0.001-0.5 Molar (M) |
| | formyl pyrrolidine | About 0.001-0.5 Molar (M) |
| | formyl piperidine | About 0.001-0.5 Molar (M) |
| | formyl morpholine | About 0.001-0.5 Molar (M) |
| Sugars, sugar | α-cyclodextrin | About 0.001-0.05 Molar (M) |
| polymers, and | maltodextrin | About 0.1-30% w/vol |
| sugar alcohols | trehalose | About 0.1-30% w/vol |
| | D-threitol | About 0.1-30% w/vol |
| | sorbitol | About 0.1-30% w/vol |
| | xylitol | About 0.1-30% w/vol |

Additives used in an in vitro TXTL reaction may or may not align with conditions from in vivo experiments. For example, macromolecular crowding is known as an important agent within cells. Macromolecular crowding helps to stabilize proteins in their folded state by varying excluded volume—the volume inaccessible to the proteins due to their interaction with macromolecular crowding agents. This is critical to cells; for example, E. coli cytoplasm contains 300-400 mg/mL of macromolecules. From this, it can be inferred that emulating the cell's behavior, such as done for the Cytomin™ system, can optimize TXTL reaction capability. However, it has since been shown that crowding from other non-natural effectors, such as polyethylene glycol, are equally effective at implementing TXTL reactions, as utilized in (Sun et al., 2013). Therefore, from in vivo findings alone it may be difficult to predict what additives can improve in vitro TXTL activity.

Provided hereunder in the examples are exemplary assays that can be used to test the effect of various additives on the transcription and translation of non-model proteins. While only a subset of additives and a subset of non-model proteins are illustrated below, those skilled in the art will recognize that these assays can be applied to other additives and other non-model proteins.

### Slow Elongation-Rate Polymerases for Improving In Vitro Transcription and Translation

In some embodiments, slow elongation-rate polymerases can be utilized to improve in vitro transcription and translation yields. Slow elongation-rate polymerases produce mRNA slower than their native counterpart. This is particularly relevant when the polymerase utilized is derived from phage, which is historically the source of transcription in TXTL reactions (e.g., T7, SP6). These polymerases in turn are typically highly processive and have high elongation-rates.

While more mRNA produced at faster speed should be intuitively better, it has been unexpectedly shown herein that slow elongation-rate polymerases can improve expression of genes, especially non-model genes. By using slow elongation-rate polymerases that retain high processivity, less amounts of mRNA for translation are transcribed within a unit time, compared to the native polymerases. However, unexpectedly, translation and coupling are improved. Without wishing to be bound by theory, this is believed to be due to a better match of translation with the native host production of mRNA than the native polymerase. While counterintuitive, better protein yield is observed. Therefore, polymerases that match the elongation rate of the native host organism can be used to improve in vitro transcription and translation. In E. coli the native elongation-rate is about 30 nt/s, while the T7 RNA polymerase native elongation-rate is about 240 nt/s.

In some embodiments, the amount of lower elongation-rate polymerases to add can be, e.g., between about 0.1 nM to 10 μM, depending on the amount of transcription products to be produced.

In some embodiments, an in vitro TXTL system can be supplemented with RNAP that is homologous to the host organism(s) from which the lysate is derived. This allows for transcriptional activity to be supplemented, if transcriptional activity is rate-limiting. For example, if a lysate prepared from one or multiple non-model host(s) is prepared, the amount of functional native polymerase in the reaction may be rate-limiting and/or a strong-strength native promoter unit used to drive the native polymerase may be unknown. This is the case in TXTL made from E. coli, where identification of a strong OR2-OR1-Pr promoter is necessary to drive efficient native transcription, as described in (Shin & Noireaux, 2010) and incorporated by reference. In the non-model host(s), a weak native promoter can be boosted in strength by supplementing the reaction with more native RNAP. Alternately, if the native RNAP is degraded and/or inactive through the TXTL preparation process, functional native RNAP can be supplemented that is produced externally to the TXTL reaction.

In some embodiments, the RNAP is not native (e.g., heterologous) to the host organism(s) from which the lysate is derived. This RNAP may produce mRNA that is compatible with native translation, and may emulate the RNAP from the host. The polymerase can be chosen to best encourage coupling with the downstream ribosome in the TXTL system, taking into consideration speed, processivity, and other biochemical factors as described in (Proshkin, Rahmouni, Mironov, & Nudler, 2010). The polymerase may require the use of its cognate promoter (rather than the promoter from the host TXTL system). The ideal polymerase has a slow elongation-rate while maintaining high processivity. This allows for the simplicity of using a high-expressing polymerase, without the need to either identify promoters that respond to the host native polymerase or optimize host polymerase expression. In some embodiments, this polymerase may have additional properties that encourage coupling that are not rate-related, such as additives that affect transcriptional and/or translational regulation.

In some embodiments, the RNAP supplied can originate from thermophiles or psychrophiles. These organisms are more likely to have stable RNAP that can be used heterologously in TXTL systems. If the elongation rate of the RNAP from a thermophile or psychrophile is too high, the TXTL reaction can be run at a non-optimal growth temperature for the RNAP's sourced thermophile or psychrophile in order to slow the elongation-rate of the RNAP.

In some embodiments, the RNAP supplied to the TXTL reaction can be engineered or synthetic. This engineered RNAP may be a variant of a naturally-occuring RNAP that is found to be effective at driving efficient transcription in the TXTL system. This includes variants of the RNAP from which the lysate is derived, as well as heterologous RNAPs, such as phage RNAPs and thermophile or psychrophile RNAPs, without exclusion. In some embodiments, the RNAP can be engineered either by rational design and/or directed evolution to have slow elongation-rate and high processivity.

In some embodiments, the RNAP supplied to the TXTL reaction can be provided as a purified protein. This protein can be produced heterologously in an expression host (e.g., E. coli, yeast, etc.) or in a separate in vitro reaction(s) and then purified in an active form and added to the TXTL reaction directly preceding the reaction start time or added to the lysate after preparation. It can also be produced synthetically. In some embodiments, the RNAP is directly expressed in the cell-free reaction. Nucleic acids that encode for the RNAP can be supplied to the TXTL reaction under a expressible promoter to produce RNAP for use in the same TXTL reaction.

In some embodiments, the TXTL reaction can be further supplied with nucleic acids containing a promoter that is recognized by the provided slow elongation-rate RNAP. This is important to drive the reaction of the desired protein and/or product to be made in the TXTL reaction. By utilizing a known promoter recognized by the supplied RNAP, one can titrate the transcription of the desired product. This is particularly important for non E. coli TXTL systems and/or systems made from non-model hosts where native transcriptional regulation may not be known and/or strong promoters are not identified. The mRNA produced can then be linked to native translation or to an orthogonal translation machinery.

### Exogenous Ribosomes for Improving In Vitro Transcription and Translation

In some embodiments, ribosomes can be supplemented to the TXTL reaction so as to further encourage transcriptional and translational coupling and protein yield. As transcription and translation are closely tied, there may be imbalances between the two, specifically in lysate-based systems where mismatch can occur from growth conditions, harvesting conditions, harvesting method, among other properties. These mismatches can be observed in cell-free reactions, as demonstrated in (Siegal-Gaskins, Tuza, Kim, Noireaux, & Murray, 2014) and incorporated by reference. To relieve this mismatch, ribosomes can be supplied exogenously in, e.g., purified form. Without wishing to be bound by theory, it is believed that doing so can relieve transcription and translation imbalance and facilitate coupling, which involves the interaction of a critical mass of ribosomes to polymerases. In some embodiments, along with exogenous ribosomes added, Magnesium and optionally ATP can also be added at a molar ratio between about 1 to 100 to 1 to 10000 of added ribosome concentration to Magnesium and optionally ATP.

In some embodiments, ribosomes added can be sourced from the host organism(s) from which the lysate is derived or can be sourced from a different organism. Ribosomes added can be heterologously produced and isolated, produced in vitro in a separate reaction, or produced synthetically. For example, for a Streptomyces spp. TXTL reaction, Streptomyces ribosomes can be heterologously produced in E. coli or yeast, purified, and added back into a Streptomyces TXTL reaction. These ribosomes may also be effective in an organism similar to Streptomyces spp., such as another actinomycete. It should be noted that while ribosomes are highly conserved, the machinery of divergent species may not be conserved enough to be cross-compatible. For example, tRNAs from the host may not recognize the exogenously supplied ribosome, or regulation of the exogenously supplied ribosome may be hindered. Therefore, ribosomes should be tested beforehand in an assay similar to those shown in the examples to ensure compatibility. Ribosomes from less divergent species will have higher likelihoods of success as additives. In some embodiments, additional additives to enable ribosome activity can be added (e.g., tRNAs, regulatory proteins such as Rqc2, eIF, RPGs, etc . . . ) to produce a functional ribosomal translation system. Ribosomes added can also be further engineered to provide advantageous properties, such as incorporation of non-standard amino acids, L- and/or D-form chemical matter, or more efficient translation.

In some embodiments, the orthogonal or complementary translation system can be linked to the suppled transcriptional system. This linkage provides an environment to conduct highly-efficient coupled TXTL reactions, but also utilize advantages that come from protein production in a lysate environment, such as the presence of necessary and/or beneficial known and/or unknown cofactors.

### EXAMPLES

### Example 1. DMSO in a TXTL System Helps Expression of Some Genetic Elements but Not Others, and Only Modulates Transcription

Dimethyl sulfoxide (DMSO) is a reagent often used in polymerase chain reactions (PCR) to avoid secondary structure formation in primers, and hence it increases PCR yields. Additionally, DMSO has also been shown to help in the denaturation of mRNA. The effect of DMSO is on transcription.

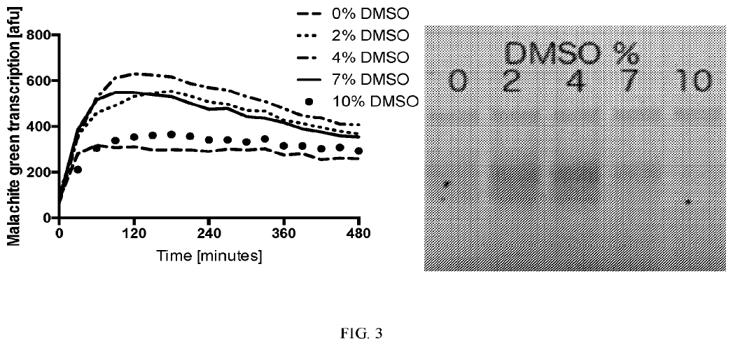

We determined whether DMSO enhanced the expression of metagenomic and/or non-model genes. We first expressed a metagenomically derived gene, lazC, (773 SEQ ID NO: 1), under a sigma70 reporter and UTR1 RBS, in a E. coli TXTL system produced by methods described in (Sun et al., 2013). This sequence has Malachite-green (Mg) aptamer, which we used to track transcription, as described in (Siegal-Gaskins et al., 2014) and incorporated by reference. The setup conditions are: 30% eAC27 E. coli lysate, 30% energy solution buffer, 30 mM Mg-dye, 1% FloroTect™, gamS, and DMSO, where lazC is run at 16 nM and Mg-aptamer is tracked kinetically in a plate-based spectrophotometer (e.g., Biotek H1, Biotek Synergy 2) as well as endpoint expression after more than 8 hours at 29 Celsius by running a SDS-PAGE gel and detection of FloroTect™ fluorescence. As shown in FIG. 3, a moderate amount of DMSO (e.g., 2%-7%) enhanced Mg-aptamter transcription efficiency, thereby improving transcription. When the same samples are run on a SDS-PAGE gel, this also leads to improvements in production of lazC protein. This shows that DMSO can affect protein yields of some genes in a transcriptional manner.

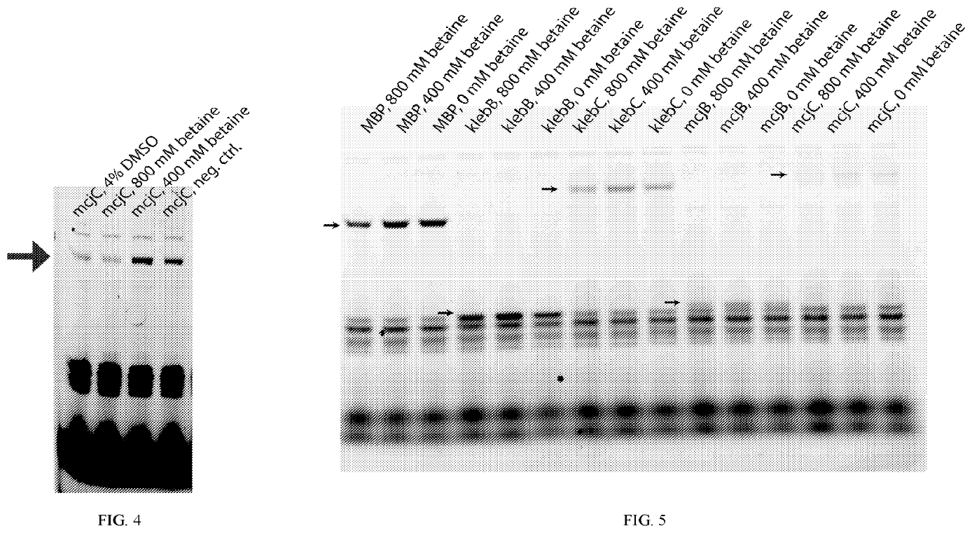

However, DMSO does not universally help cell-free transcription and translation for all genes. In FIG. 4, we expressed 6 nM of a sigma70-mcjC linear DNA construct (728, SEQ ID NO: 2) in a E. coli TXTL system produced by methods described in (Sun et al., 2013). The setup conditions are: 30% eAC28 E. coli lysate, 33% energy solution buffer, 30 mM Mg-dye, 1% FloroTect™, 20 μg/ml gamS, and additives DMSO at 4% working concentration, betaine at 400-800 mM, or nothing (negative control). After expressing more than 8 hours at 29 Celsius, we run a 4-12% SDS-PAGE gel loaded with 2 μL of each reaction and detection of FloroTect™ fluorescence. Visually, added DMSO preforms no better than the negative control, and potentially worse than other additives. While DMSO can help some genes, DMSO also does not impact and can hurt expression of other genes.

### Example 2. Other Additives can Assist TXTL Expression via Different Mechanisms than DMSO

Betaine in E. coli TXTL System Helps Expression of Some Genetic Elements

In FIG. 4, we also show that 400 mM betaine helped improve expression of sigma70-mcjC, as this amount is below the toxicity level to TXTL but high enough to provide an expression effect. The mechanism by which betaine acts is different from DMSO, as DMSO does not help sigma70-mcjC expression.

We then ran betaine across multiple genes with different activities and show that the effect is not limited to mcjC. We utilized the same conditions as described for FIG. 4, but only tested betaine at 0 mM, 400 mM, and 800 mM and run 6 nM linear DNA of sigma70(lacO1)-MBP (1066, SEQ ID NO: 3), sigma70-klebB (938, SEQ ID NO: 4), sigma70-klebC (939, SEQ ID NO: 5), sigma70-mcjB (727, SEQ ID NO: 6), and sigma70-mcjC (728, SEQ ID NO: 2). In FIG. 5, we saw that betaine helped klebB, klebC, mcjB, and mcjC at 400 mM concentrations but has no appreciable effect on MBP. This confirms that betaine can help the expression of many genes in the TXTL system.

### Betaine in Streptomyces TXTL System Helps Expression of Some Genetic Elements

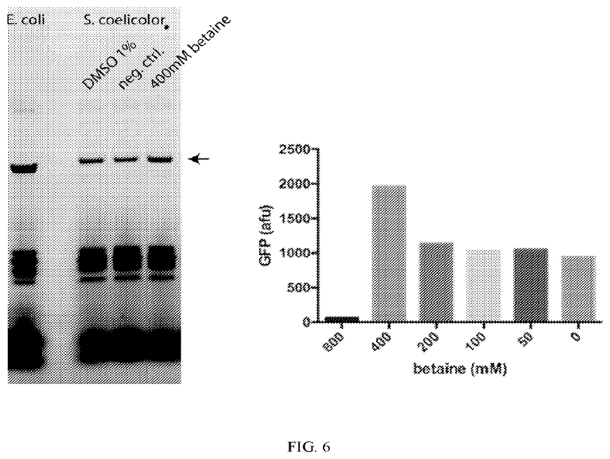

We also demonstrate betaine improving expression of additional genes in a non-E. coli, Streptomyces coelicolor TXTL system. A S. coelicolor TXTL system was prepared according to (Li, Wang, Kwon, & Jewett, 2017), where in lieu ISP2 medium was used for growth, washed twice in cold Wash Buffer 1 (10 mM HEPES-KOH pH 7.5, 10 mM magnesium glutamate, 1 M potassium glutamate, 1 mM DTT), once in Wash Buffer 2 (50 mM HEPES-KOH pH 7.5, 10 mM magnesium glutamate, 50 mM potassium glutamate, 1 mM DTT), and once in Wash Buffer 3 (50 mM HEPES-KOH pH 7.5, 10 mM magnesium glutamate, 50 mM potassium glutamate, 1 mM DTT, 10% (v/v) glycerol), and lysis was done using a French press at 12,000 psi. The energy solution is from (Sun et al., 2013). The setup conditions are: 30% eSC3 S. coelicolor lysate, 34% energy solution buffer, 1% FloroTect™, and additives DMSO at 1% working concentration, betaine at 400 mM, or nothing (negative control). After expressing more than 8 hours at 29 Celsius, we run a 4-12% SDS-PAGE gel loaded with 2 μL of each reaction and detection of FloroTect™ fluorescence. In FIG. 6, left, we show the expression of 15 nM of a linear DNA MBP construct variant (1350, SEQ ID NO: 7), in S. coelicolor TXTL which produces more protein with betaine than without betaine. For comparison, a E. coli TXTL reaction expressing a different MBP variant is provided for reference. We also conduct a TXTL reaction like the above, but utilizing betaine in lieu of DMSO at 0-800 mM to track GFP expression using a plate reader, and including 0.1 mg/ml T7 RNAP working concentration. In FIG. 6, right, we show that 6 nM linear DNA expressing GFP (linear version of Addgene 40019 amplified with SEQ. ID. 14 and SEQ. ID. 15 but utilizing the T7 promoter sequence in SEQ. ID NO: 12) also produces more GFP at betaine concentration of 400 mM. This shows that the effect of betaine is generalizable across multiple cell-free systems.

### Example 3. Improving In Vitro Transcription and Translation by Utilizing Slow Elongation-Rate Polymerases Improves Expression of Many Genes

T7 Polymerase Produces Less Protein than Native Polymerase Despite Higher Transcript Production.

We first construct a library of T7 promoters varying in strength each expressing GFP in cell-free systems. These are numbered from 695, a sigma70 control as plasmid (SEQ ID NO: 8) and linear, to 688 (SEQ ID NO: 9), 696 (SEQ ID NO: 10), 697 (SEQ ID NO: 11), 698 (SEQ ID NO: 12), 699 (SEQ ID NO: 13) as T7 promoter variants, as plasmid and linear, where the sequence listing provides the promoter region. Each plasmid is constructed by cloning the sequence between sites “GCAT” and “AAGC” (position 1 to position 69 in SEQ ID NO: 8) using standard molecular biology techniques. Linear DNA is made by amplifying each ligation product proceeding the production of the plasmid with primers 30810f (SEQ ID NO: 14) and 30810r (SEQ ID NO: 15) with polymerase chain reaction (PCR), as described in (Sun, Yeung, Hayes, Noireaux, & Murray, 2014) and incorporated by reference.

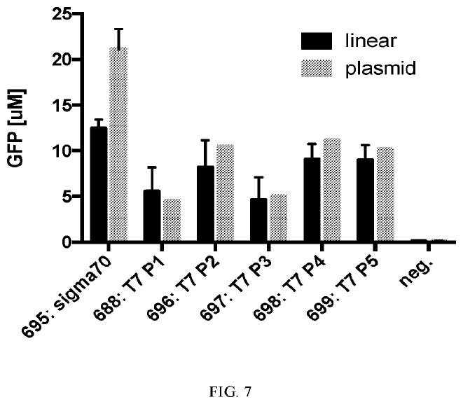

Each sequence is tested for its expression of GFP in the same reaction, done with two repeats. Conditions are: E. coli lysate eZS4/bZS4 at 25%/25% total reaction prepared as described in (Niederholtmeyer et al., 2015), gamS at 3.5 μM, and NEB T7 M0251L 12 Units/mL working from custom 30x stock, where all linear DNAs are tested at 16 nM and plasmid DNA at 8 nM and cell-free expression is measured after 10 hours. In FIG. 7, T7 expression measured by GFP production is less than sigma70 expression in all cases when linear DNA and plasmid DNA is compared. This is despite T7's higher processivity. Expressing T7-driven coding sequences from linear DNA does not relieve the expression deficit, suggesting it is not due to T7's propensity to make multiple strands of mRNA. Results are not explained by mRNA sequence or structure. The secondary structure transcript from T7 and sigma70 are identical as all have the same transcription start site. All also share the same ribosome binding site.

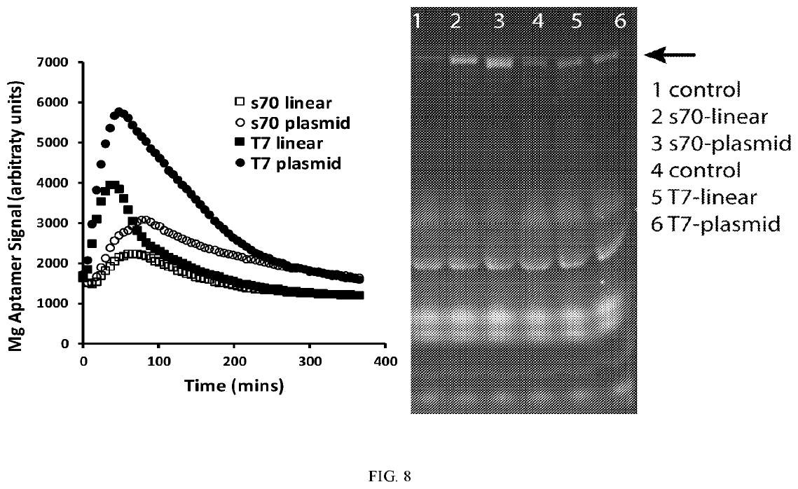

We then show that for many non-model proteins, we see weaker overall expression under a T7 expression vector compared to a sigma70 expression vector in TXTL. In FIG. 8, we express a metagenomic coding sequence from sigma70 (SEQ ID NO: 16) and from T7 (SEQ ID NO: 17). This sequence has Malachite-green (Mg) aptamer, which we used to track transcription. The setup conditions are: 30% eAC27 E. coli lysate, 30% energy solution buffer, 30 mM Mg-dye and/or FloroTect™, NEB T7 M0251L 12 Units/mL working from custom 30x stock, and gamS. The coding sequence is run at 16 nM linear and 8 nM plasmid and tracked at 590/35 ex 645/166 em in a Biotek Synergy 2. Plotted in FIG. 8, left, are Mg-aptamer signal for both the linear and plasmid sigma70 and T7 expressed versions of the coding sequence. The T7 expressed version produces more mRNA than the sigma70 expressed version, as the Mg-aptamer tag is placed on the 3′ end of the transcript and should capture total mRNA production. However, the corresponding SDS-PAGE gel on FIG. 8, right, shows that the T7 expressed version produces less protein than the sigma70 expressed version. Therefore, there is a generalizable advantage of the slower sigma70 polymerase over the faster T7 RNAP.

### Slowing Polymerase Elongation-Rate can Encourage Coupling and Produce Increased Protein.

To encourage coupling, we will engineer and/or supply polymerases with reduced elongation rates that match transcription rates with native translation rates.

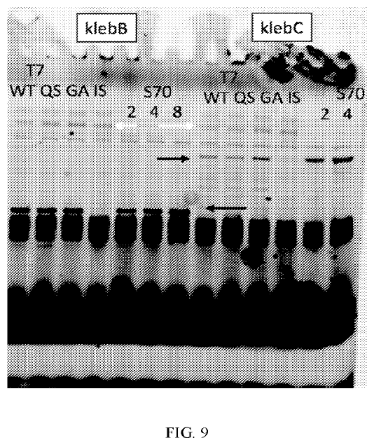

To test matching of transcription to translation, we utilized T7 RNAP variants from (Bonner et al., 1994; Makarova, Makarov, Sousa, & Dreyfus, 1995), incorporated by reference, that are known to have slower processivity in vitro than the wildtype form. Specifically, we tested four variants: a wildtype (240 nt/s elongation rate, 0.94 processivity), a Q649S variant (160 nt/s elongation rate, 0.88-0.91 processivity), a G645A variant (90 nt/s elongation rate, 0.81-0.87 processivity), and a 1810S variant (40 nt/s elongation rate, 0.70-0.75 processivity). The native E. coli polymerase elongation rate is 30 nt/s with high processivity. In one experiment, we expressed two metagenomic proteins, klebB and klebC, as sigma70 and T7 constructs (sigma70-klebB, 938, SEQ ID NO: 4, T7-klebB, 1204, SEQ ID NO: 18, sigma70-klebC, 939, SEQ ID NO: 5, T7-klebC, 1205, SEQ ID NO: 19). The T7 RNAP variants are expressed off of linear DNA as sigma70-T7WT (1381, SEQ ID NO: 20), and variants mutated in the CDS as Q649S, G645A, and I810S with the same structure as 1381. In samples with T7, T7 RNAP mutants are expressed at 1.5 nM for the WT variant and 1 nM for the mutants, and linear T7-klebB and klebC are expressed at 4 nM. In samples with sigma70, sigma70-klebB and klebC are expressed at 2 nM, 4 nM (and 8 nM for klebB). Expression was done with E. coli TXTL eCA1 and bACn4 produced by methods described in (Sun et al., 2013), with FloroTect™ and gamS. Reactions were expressed overnight and detected using a SDS-PAGE gel. In FIG. 9, the results of the TXTL expression are shown, where the white arrow represents the expected size of the produced protein and the black arrow represents production of the T7 RNAP or mutant thereof. For klebC, the expression from the T7 RNAP G645A variant is superior to the WT variant. These are still less than sigma70-klebC. For klebB, expression from all variants except for the I810S variant are similar. This indicates potential differences due to polymerase elongation rate.

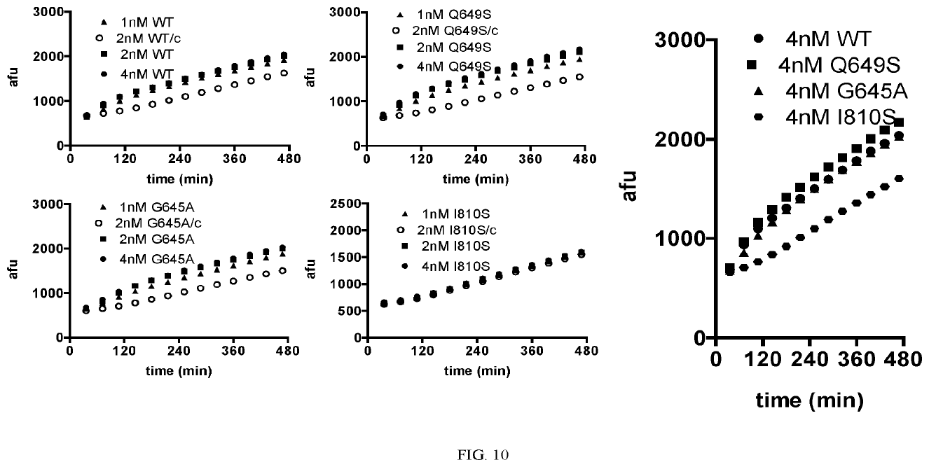

We also test T7 RNAP variants against a T7-MBP (1338, SEQ ID NO: 21) and T7-MBP-FlAsH (“CCPGCC” tag) gene (1339, SEQ ID NO: 22). Here, we exprss the linear sigma70-T7WT and Q649S, G645A, and I810S variants as described previously, at 1 nM, 2 nM, and 4 nM concentrations. These are expressed with 4 nM of either linear T7-MBP or T7-MBP- FlAsH. Expression was done with E. coli TXTL eCA1 and bACn4 produced by methods described in (Sun et al., 2013), with FlAsH reagent at 20 μM and gamS. Plotted is detection of FlAsH at 428/20 em and 528/20 ex in a Biotek Synergy 2, where FlAsH binding to the tag is kinetically tracked to protein production. We see in FIG. 10, left, controls where 2 nM vs 4 nM T7 RNAP WT or mutant does not change final MBP detection—this tells us transcription is saturated. Then, comparing the 4 nM case on FIG. 10, right, we can see that expression of MBP—FlAsH is best with Q649S over the WT, while G645A is comparable and I810S worse. The 4 nM case matches the 2 nM case well. This shows that different polymerase elongation rates can lead to improvements in expression, again dependent on gene and polymerase mutant.

While a polymerase with slower elongation rate should cause transcription and translation to improve, additional additives can also be added to further promote coupling and protein yield. Such additives may include metals (e.g., manganese, magnesium, cobalt), proteins (e.g., chaperones,), and chemical stabilizers (e.g., betaine, polyethylene oxide), among others. These additives can be used in combination with an engineered and/or supplemented natural polymerase.

Polymerases can be Rationally Designed and/or Evolved to be Slow Elongation-Rate.

To engineer a suitable slow elongation-rate polymerase, we can rely on rational design. In the specific case of T7 RNAP, as described in (Sousa et al., 1993) and incorporated by reference, rational mutations will be made in the active site of the enzyme and then tested in vitro for elongation-rate and processivity as described in (Makarova et al., 1995). Furthermore, each mutated T7 RNAP can be tested in the methods described herein in high-throughput format for MBP-FlAsH, MBP, and other FlAsH and non- FlAsH tagged genes, where the new T7 RNAP variant is tested similarly relative to a wild-type control. We can further engineer the polymerase by directed evolution. Continuing with the example of T7 RNAP, T7 RNAP has been shown to be engineered using phage-assisted continuous evolution by (Esvelt et al., 2011), incorporated by reference. Selection pressure for slower elongation rate but equal processivity to wildtype can be applied and multiple cycles of continuous evolution can be conducted to produce a T7 RNAP with desired properties. Other directed evolution methods can be applied, such as described in (Renata et al., 2015), incorporated by reference.

### Example 4. Supplementing Ribosomes to the Reaction Encourages Transcriptional and Translational Coupling and Protein Yield

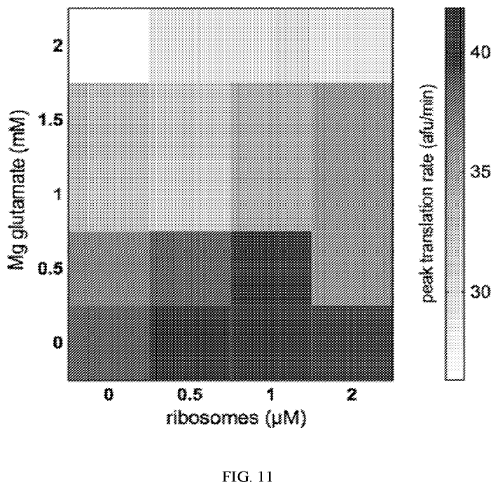

To demonstrate ribosome addition helping TXTL reactions, we show the addition of purified 70S ribosomes to a E. coli TXTL system. We utilize purified ribosomes from E. coli B strain (New England Biolabs, P0763S, 13.3 μM). These ribosomes are stored in a buffer of 20 mM HEPES-KOH pH 7.6, 10 mM Mg-acetate, 30 mM KCl, and 7 mM b-mercaptoethanol. Those skilled in the art will recognize that the buffer can introduce large toxicity effects into TXTL reactions, especially glycerol in the case of E. coli TXTL reactions; however, the chemicals listed here are not toxic from internal testing and from data in (Sun et al., 2014), incorporated by reference. Expression was done with E. coli TXTL eAC28 and bACn5 produced by methods described in (Sun et al., 2013), with 0-2 μM working concentration of P0763S NEB Ribosomes and 0-2 mM working concentration of Mg-glutamate. 8 nM of a sigma70-GFP control plasmid (Addgene #40019) was supplied, and expression was tracked kinetically by fluorescence for 12 hours. Peak translation rate was determined by taking the slope of arbitrary fluorescence units (afu) between each time point (data was collected at 6 min intervals). Peak translation rate is the highest rate observed. Typically the highest rates are seen early in a TXTL reaction. As shown in FIG. 11, which plots peak translation rates per minute, there is a direct correlation between increased ribosomes (and corresponding increased Mg concentration) and signal above the 0 mM added ribosomes, 0 mM added Mg-glutamate case. This demonstrates that additional ribosomes are able to increase peak production of protein, and encourage better translation and coupling. ATP can also be added at equimolar concentrations of Magnesium to improve expression.

### REFERENCES

Bonner, G., Lafer, E. M., & Sousa, R. (1994). Characterization of a set of T7 RNA polymerase active site mutants. The Journal of Biological Chemistry, 269(40), 25120-25128.

Buntru, M., Vogel, S., Spiegel, H., & Schillberg, S. (2014). Tobacco BY-2 cell-free lysate: an alternative and highly-productive plant-based in vitro translation system. BMC Biotechnology, 14(1), 37. http://doi.org/10.1186/1472-6750-14-37

Chiao, A. C., Murray, R. M., & Sun, Z. Z. (2016). Development of prokaryotic cell-free systems for synthetic biology. bioRxiv, 048710. http://doi.org/10.1101/048710

Esvelt, K. M., Carlson, J. C., & Liu, D. R. (2011). A system for the continuous directed evolution of biomolecules. Nature, 472(7344), 499-503. http://doi.org/10.1038/nature09929

Golomb, M., & Chamberlin, M. (1974). Characterization of T7-specific Ribonucleic Acid Polymerase IV. RESOLUTION OF THE MAJOR IN VITRO TRANSCRIPTS BY GEL ELECTROPHORESIS. The Journal of Biological Chemistry, 249(9), 2858-2863.

Hansen, M. M. K., Ventosa Rosquelles, M., Yelleswarapu, M., Maas, R. J. M., van Vugt-Jonker, A. J., Heus, H. A., & Huck, W. T. S. (2016). Protein Synthesis in Coupled and Uncoupled Cell-Free Prokaryotic Gene Expression Systems. ACS Synth Biol, 5(12), 1433-1440. http://doi.org/10.1021/acssynbio.6b00010

Jewett, M. C., & Swartz, J. R. (2004). Mimicking the Escherichia coli cytoplasmic environment activates long-lived and efficient cell-free protein synthesis. Biotechnol Bioeng, 86(1), 19-26. http://doi. org/10.1002/bit. 20026

Kelwick, R., Webb, A. J., MacDonald, J. T., & Freemont, P. S. (2016). Development of a Bacillus subtilis cell-free transcription-translation system for prototyping regulatory elements. Metab Eng, 38, 370-381. http://doi.org/10.1016/j.ymben.2016.09.008

Kim, D.-M., & Swartz, J. R. (2001). Regeneration of adenosine triphosphate from glycolytic intermediates for cell-free protein synthesis. Biotechnol Bioeng, 74(4), 309-316. http://doi.org/10.1002/bit.1121

Li, J., Wang, H., Kwon, Y.-C., & Jewett, M. C. (2017). Establishing a high yielding Streptomyces-based cell-free protein synthesis system. Biotechnol Bioeng. http://doi.org/10.1002/bit.26253

Lutz, R., & Bujard, H. (1997). Independent and Tight Regulation of Transcriptional Units in Escherichia Coli Via the LacR/O, the TetR/O and AraC/I1-I2 Regulatory Elements, 25(6), 1203-1210. http://doi.org/10.1093/nar/25.6.1203

Makarova, O. V., Makarov, E. M., Sousa, R., & Dreyfus, M. (1995). Transcribing of Escherichia coli genes with mutant T7 RNA polymerases: stability of lacZ mRNA inversely correlates with polymerase speed. Proceedings of the National Academy of Sciences of the United States of America, 92(26), 12250-12254. http://doi.org/10.1073/pnas.92.26.12250

McClure, W. R., & Chow, Y. (1980). [11] The kinetics and processivity of nucleic acid polymerases. In Enzyme Kinetics and Mechanism—Part B: Isotopic Probes and Complex Enzyme Systems (Vol. 64, pp. 277-297). Elsevier.

Niederholtmeyer, H., Sun, Z., Hori, Y., & Yeung, E. (2015). Rapid cell-free forward engineering of novel genetic ring oscillators. eLife. http://doi.org/10.7554/eLife.09771.001

Proshkin, S., Rahmouni, A. R., Mironov, A., & Nudler, E. (2010). Cooperation between translating ribosomes and RNA polymerase in transcription elongation. Science, 328(5977), 504-508. http://doi.org/10.1126/science.1184939

Renata, H., Wang, Z. J., & Arnold, F. H. (2015). Expanding the enzyme universe: accessing non-natural reactions by mechanism-guided directed evolution. Angewandte Chemie International Edition, 54(11), 3351-3367. http://doi.org/10.1002/anie.201409470

Shin, J., & Noireaux, V. (2010). Efficient cell-free expression with the endogenous E. Coli RNA polymerase and sigma factor 70. J Biol Eng, 4(1), 8-9. http://doi.org/10.1186/1754-1611-4-8

Shin, J., & Noireaux, V. (2012). An E. coli Cell-Free Expression Toolbox: Application to Synthetic Gene Circuits and Artificial Cells. ACS Synth Biol, 1(1), 29-41. http://doi.org/10.1021/sb200016s

Siegal-Gaskins, D., Tuza, Z. A., Kim, J., Noireaux, V., & Murray, R. M. (2014). Gene circuit performance characterization and resource usage in a cell-free “breadboard.”ACS Synth Biol, 3(6), 416-425. http://doi.org/10.1021/sb400203p

Sousa, R., Chung, Y. J., Rose, J. P., & Wang, B.-C. (1993). Crystal structure of bacteriophage T7 RNA polymerase at 3.3 Å resolution. Nature, 364(6438), 593-599. http://doi.org/10.1038/364593a0

Sun, Z. Z., Hayes, C. A., Shin, J., Caschera, F., Murray, R. M., & Noireaux, V. (2013). Protocols for Implementing an Escherichia Coli Based TX-TL Cell-Free Expression System for Synthetic Biology. Journal of Visualized Experiments, e50762(79), e50762-e50762. http://doi.org/10.3791/50762

Sun, Z. Z., Yeung, E., Hayes, C. A., Noireaux, V., & Murray, R. M. (2014). Linear DNA for Rapid

Prototyping of Synthetic Biological Circuits in an Escherichia coli Based TX-TL Cell-Free System. ACS Synth Biol, 3(6), 387-397. http://doi.org/10.1021/sb400131a

Thompson, J., Rae, S., & Cundliffe, E. (1984). Coupled transcription—translation in extracts of Streptomyces lividans. Molecular and General Genetics MGG, 195(1-2), 39-43. http://doi.org/10.1007/BF00332721

### EQUIVALENTS

The present disclosure provides among other things cell-free systems and use thereof. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and sequences mentioned herein are hereby incorporated by reference in their entirety as if each individual publication or patent was specifically and individually indicated to be incorporated by reference.

SEQUENCE LISTING:

SEQ ID NO: 1
>773 sigma70-lazC region
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGTCTGATCCTGCTGATGGGCGTGGGGCCGTAACGGCGTGGGACGTGGTATTGTA
TCACTATCGCCCGGATAAGGCTCGCGCATTACGCGAGGCAGTTCTTCCATTGGCCCGTCAGGCTGCTGCCGAAGGATTGG
CCGCACACGTGGAGCGCCACTGGCGTTTCGGCCCACATCTTCGCCTGCGCTTGCGTGGTCCTGAGGCGCGTGTTGCTGGG
GCCGCCCAGCGTGCTGCAGAGGCGCTGCGCGCATGGGCAGCGGCACACCCGTCAGTAGCCGATCGCTCTGATGAGCAATT
ATTAGCCGAAGCCGCAGTAGCCGGACGTGCAGAACTGATTGCCCCACCCTATGCTCCCCTTGTTCCAGATAACACCGTTG
TTGCTGCGCCAGCAGACCGCTCGGCGGAGGACGCACTTCGTGCGTTAATTGGTGCTGAATCCGCTGAGTTACGTGAGGAG
TTGCTTCGTACGGGTTTACCGGCATTGGACTCCGCTTGCCACTTCCTGGGGGCGCATGGGGATACTCCCCAAGCACGCGT
ACAATTAGTGGTAACAGCGCTTGCTGCCCATGCCACGGCCCACCCCGACGGACTGGTTGGAGCCCATTACTCTGTGCTGA
GTCATCTTGAGGATTTCTTAGTTCACGAAGATCCCGACGGGAGTCTGCGTGCAGCCTTCGAACGCCGTTGGGAACAGTCG
GGTCGCGCCGTCACGGCATTGGTTGGTCGTATTGCCGACGGGGGGGCGCGTGATTGGGAACGTGATTGGGCACACTGGTC
GGCGACTGCTTGGTCTTTGGCCGAGCGTCGCTTAACAGCGGGCGCCGATCTGGGTGGTCGTCACGCGGAGTACCGTGAAC
GCGCAGAAGCGCTTGGCGACCCTGCAACAGCCGAACGTTGGAACGCGGAACTGCGCACCCGTTACAGCGAGTTTCATCGT
ATGTTACAGCGTGCGGACCCTGATGGACGCATGTGGCACCGCCCCGACTACTTGATCAATCGTGCGGGAACCAATGGTTT
GTACCGCTTGTTAGCTATCTGCGATGTACGCCCTATGGAACGCTATCTTGCAGCGCACTTGCTGGTACGCAGTGTTCCGG
AGCTTACAGGGCATCGTTGGCAGACTCTGCTGGGGGCTGCAGAGCAACCGGGCGGCCCTGAGCAGAGTGGCGCGGCTGGC
GCTACGGGCGGGGCTGGCCGTACCAAACTGGAAGGTGCTGCCTGATGAATAACTGAATAGGGGATCCCGACTGGCGAGAG
CCAGGTAACGAATGGATCCCCGAGCTCGAGCAAAGCCCGCCGAAAGGCGGGCTTTTCTGTCCTTGAGAGTCGGGCATTGT
CTTCGCTCCTTCCGGTGGGCGCGGGGCATGACTATCGTCGCCGCACTTATGACTGTGTTCTTTATCAT
SEQ ID NO: 2
>728 sigma70-mcjC
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGGAAATATTTAATGTCAAGTTAAATGATACTTCAATTAGAATTATTTTCTGTAA
AACGCTTTCTGCCTTCCGGACAGAAAATACCATCGTTATGCTCAAAGGAAAAGCAGTTTCAAATGGCAAACCTGTATCCA
CAGAGGAGATTGCCAGAGTAGTGGAAGAAAAAGGTGTTTCAGAAGTAATAGAAAATTTAGATGGTGTTTTCTGTATCCTA
ATTTATCATTTTAATGATCTCCTTATAGGGAAAAGCATTCAATCAGGCCCCGCTCTATTTTATTGTAAAAAGAATATGGA
TATTTTTGTTTCGGATAAAATTTCTGATATCAAATTTTTGAATCCAGATATGACATTCAGTCTAAATATAACAATGGCAG
AACATTATCTGTCAGGAAATCGAATAGCAACCCAGGAATCACTAATCACTGGCATTTACAAAGTAAATAATGGTGAGTTT
ATAAAATTTAATAATCAGTTGAAACCTGTGCTACTTCGTGATGAGTTTAGTATTACCAAAAAGAACAATTCAACTATCGA
CAGTATCATTGATAATATTGAGATGATGCGGGATAATAGAAAAATAGCCCTATTATTTTCCGGAGGATTGGATTCTGCAT
TAATTTTTCACACACTTAAAGAATCAGGTAACAAATTCTGCGCTTATCATTTTTTTTCTGATGAATCTGATGACAGTGAA
AAGTATTTTGCTAAGGAATACTGTTCAAAATATGGAGTTGATTTTATATCTGTTAATAAAAACATCAACTTTAATGAAAA
ACTTTATTTCAATTTAAATCCTAATAGTCCGGACGAAATCCCTTTGATATTTGAACAGACAGATGAAGAAGGTGAAGGTC
AGCCCCCCATAGACGATGATTTATTATATCTATGTGGTCACGGTGGAGATCATATTTTCGGACAAAATCCTTCAGAACTT
TTTGGCATTGATGCATATCGAAGTCATGGCTTGATGTTTATGCATAAAAAAATAGTAGAATTTTCCAATCTCAAGGGAAA
GAGATATAAAGATATCATATTTTCAAATATTTCCGCATTCATTAATACATCCAACGGATGTTCTCCAGCAAAGCAAGAGC
ACGTATCAGATATGAAACTTGCCTCTGCTCAGTTTTTTGCAACTGATTATACAGGAAAAATTAATAAACTAACTCCATTC
CTGCATAAAAATATTATCCAGCATTATGCTGGCTTACCAGTTTTTAGTCTATTTAACCAGCACTTTGATCGTTATCCCGT
TCGTTATGAAGCGTTTCAACGATTTGGTTCAGATATTTTCTGGAAAAAAACCAAACGGTCATCTTCACAGCTAATATTCA
GAATTCTATCCGGTAAAAAGGATGAACTAGTGAATACAATAAAACAGTCAGGATTAATTGAAATATTAGGCATTAACCAT
ATTGAATTGGAAAGCATTTTGTATGAAAATACGACTACACGTCTGACAATGGAACTACCATATATACTTAACTTATACCG
TCTGGCAAAATTCATTCAACTTCAATCCATTGATTATAAAGGTTAATGAACTCAAAGCCCGCCGAAAGGCGGGCTTTTCT
GTCCTTGAGAGTCGGGCATTGTCTTCGCTCCTTCCGGTGGGCGCGGGGCATGACTATCGTCGCCGCACTTATGACTGTGT
TCTTTATCAT
SEQ ID NO: 3
>1066 sigma70(lacO1)-MBP
AAAACCGAATTTTGCTGGGTGGGCTAACGATATCCGCCTGATGCGTGAACGTGACGGACGTAACGAAGACAACCACGCAT
TGCTGTTCATAAATGTGAGCGGATAACATTGACATTGTGAGCGGATAACAAGATACTGAGCACAGCAAGCAATAATTTTG
TTTAACTTTAAGAAGGAGATATACAATGAAAATCGAAGAAGGTAAACTGGTAATCTGGATTAACGGCGATAAAGGCTATA
ACGGCCTCGCTGAAGTCGGTAAGAAATTCGAGAAAGATACCGGAATTAAAGTCACCGTTGAGCATCCGGATAAACTGGAA
GAGAAATTCCCACAGGTTGCGGCAACTGGCGATGGCCCTGACATTATCTTCTGGGCACACGACCGCTTTGGTGGCTACGC
TCAATCTGGCCTGTTGGCTGAAATCACCCCGGACAAAGCGTTCCAGGACAAGCTGTATCCGTTTACCTGGGATGCCGTAC
GTTACAACGGCAAGCTGATTGCTTACCCGATCGCTGTTGAAGCGTTATCGCTGATTTATAACAAAGATCTGCTGCCGAAC
CCGCCAAAAACCTGGGAAGAGATCCCGGCGCTGGATAAAGAACTGAAAGCGAAAGGTAAGAGCGCGCTGATGTTCAACCT
GCAAGAACCGTACTTCACCTGGCCGCTGATTGCTGCTGACGGGGGTTATGCGTTCAAGTATGAAAACGGCAAGTACGACA
TTAAAGACGTGGGCGTGGATAACGCTGGCGCGAAAGCGGGTCTGACCTTCCTGGTTGACCTGATTAAAAACAAACACATG
AATGCAGACACCGATTACTCCATCGCAGAAGCTGCCTTTAATAAAGGCGAAACAGCGATGACCATCAACGGCCCGTGGGC
ATGGTCCAACATCGACACCAGCAAAGTGAATTATGGTGTAACGGTACTGCCGACCTTCAAGGGTCAACCATCCAAACCGT
TCGTTGGCGTGCTGAGCGCAGGTATTAACGCCGCCAGTCCGAACAAAGAGCTGGCAAAAGAGTTCCTCGAAAACTATCTG
CTGACTGATGAAGGTCTGGAAGCGGTTAATAAAGACAAACCGCTGGGTGCCGTAGCGCTGAAGTCTTACGAGGAAGAGTT
GGCGAAAGATCCACGTATTGCCGCCACCATGGAAAACGCCCAGAAAGGTGAAATCATGCCGAACATCCCGCAGATGTCCG
CTTTCTGGTATGCCGTGCGTACTGCGGTGATCAACGCCGCCAGCGGTCGTCAGACTGTCGATGAAGCCCTGAAAGACGCG
CAGACTCGTATCACCAAGTAATGAATAACTGAATAGGGGATCCCGACTGGCGAGAGCCAGGTAACGAATGGATCCCCGAG
CTCGAGCAAAGCCCGCCGAAAGGCGGGCTTTTCTGTCCTTGAGAGTCGGGCATTGTCTTCGCTCCTTCCGGTGGGCGCGG
GGCATGACTATCGTCGCCGCACTTATGACTGTGTTCTTTATCAT
SEQ ID NO: 4
>938 sigma70-klebB
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGTTCAACATGACACACTACCTGCGTTTCTCCATTTACAAGAACGACCTGGTGAT
CATTGATATAAATAACGACGAATATTTCATAATGAACGATGTGAATCACGAGAATATTAGCTTGCTGACTGATGTGGAGG
AGGAACTTTTAGCCGCCGGACTTATTCAGTCACTCACCCCAATTGGAGGCGATAATGAGAATTTTTACGATGAACGCTGG
CTCCCGAGGAAGGCAATCTTACGCAAGATTAATCCTGTGCTTTTACTGATGGTTTATAGCATCTTTGTAAAGTGTAAAAA
AAACCTGGATTCTAATGGCTATTATGGTGTCATTTACAGTCTGCAGAACGTTAAAAAAAACCACCGATGGGATAAATATT
CGCCCGGTGACATTATCAATTGCTTAAACTTTATTATGCCGTTTAAACATTGCGAAAATCCTTGCCTAATCTATTCATAT
GCACTGGTTACCATGCTGAAAAAAGCTACGGGGAAAGGTACGCTGGTGGTTGGTGTTCGCACTCGTCCATTCATCAGTCA
TGCGTGGGTAGAACTCGACGGGGAAATCATCTCCGATAACATTTATTTGCGTGACAAACTGTCGGTAATCATGGAAGTGT
GATGAATAACTGAATAGGGGATCCCGACTGGCGAGAGCCAGGTAACGAATGGATCCCCGAGCTCGAGCAAAGCCCGCCGA
AAGGCGGGCTTTTCTGTCCTTGAGAGTCGGGCATTGTCTTCGCTCCTTCCGGTGGGCGCGGGGCATGACTATCGTCGCCG
CACTTATGACTGTGTTCTTTATCAT
SEQ ID NO: 5
>939 sigma70-klebC
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGTTAATTATCACTGGCAATAAAAAAAGCACGGGCGCGGATAACTATATTTGCAA
GCATAATATGTACATTTATGCGGATGAGAACTATGACACCTGGGATTACAAAGATACATACATAATCTTTAAAGGCTATT
GTTTTGATGAGGACGGTAACGGCATTGCCATCAATAAAGATAGTTTTCTCCCGGAAGTCCTGGATCGCTTGCCCGAATTT
AGTGGATTTTTCGTGCTCATCACAATCTCCAAAGACAAAACCGTTATATACAAGAGCTTAAGTCGCAACACTGATGTCTT
TTACAGCGTTGATGACAATAACTTGACCATCTCGGATAATATCAAACTGCTGAGTGAACTGCTCGGCAAAAAAACTATTG
ATCCTAAATTTTTTAGTAGCTTTGTTAATAATGCATGGGTCGCGGTGTTCCTGTCGCCGATTTCTGGAATTGAAAAAATT
AATGGTGGTTGCAAATATGTTTTTGACTCTGCTGGGGTGCACGTGTTTAAACACGCTAATTTAACGCCGACCAATAAGGA
CTTCATTGAAGTGACCTCAAATATGATCAAATCGGTTTGTAAAAATAAAAAGGTGTTTTTACATCTGTCTGGCGGGTTCG
ACTCCACGTTTATTTTTTATATACTAAAAAAAACGAACATCTTCTTCGAAGTTTATACCCATGCTCCTGACCGTTACGAT
AATGATTCAGAAGTGAATCGTGTCCGGGAACTTTGCACTAAGAATAACGTACCGTTTCGTGTTGTGAGTGGTTTTCCAGA
TATTCTCAAATCTAACAAAGAAGTGTCAATACCCTCTGACGTCAATGTGATTGAGAACGAATCCGAGGACAACCAGTACA
ACGAGCTGTTAAACAACCACGATATCGTATTCTTGAACGGCCATGGCGGGGACTGTCTATTTGTGCAAAACCCATCACTG
AAGTCCGTACACCATCGTCTGAAACACGGACGTTTGATTAAGGGTTTGTGCAACGCTTATAAACTGTGCCGCCTTAAGTA
TCTTTCTTTCACAGAGATCATCAATCCAAGAAGCCGGATTCATTGCAACAACTGGTTTAGCGACACAAAATATAAAGGTT
TCTACCAGCATCCGCTGCTGATCAACATCGATGATTCGTCACCGGAATATGACCATATTGCCAACATGCTGTACTTTATG
GAGTCACTGCCTCTGCAACTGAAGGGGGGAGCAATGATGTTCAGCCCATTTCTTATGAGCTGTGCATTTCGGGTATTTAT
GP+32ATAGGTATGACGATAATTTCTCATCCGAGCACGATCGCATTCTCGCCCGAAAAATCGCCTACAACATTGCGCATG
ATATCCAACTGTTCGATGTACGTAAACGCTCGTCCAACAATCTGCTGTTCGACTTTCTGCATAAGAATAAGGAAAAGATT
CTTTTGCTGATCAACCGAGGCTTCACACAGGGTATGGGTGAGGTAACCACCGATGATCTGAAAGAATCGTTAGAAATTAA
TACCAGTATTGGGATAGATGGTAATGCGACGAAATTCCTGAAACTGATGATGTTAAACCGCTATGCAGAAATGAATATGC
TTACGAAAGAGTAGTGAATAACTGAATAGGGGATCCCGACTGGCGAGAGCCAGGTAACGAATGGATCCCCGAGCTCGAGC
AAAGCCCGCCGAAAGGCGGGCTTTTCTGTCCTTGAGAGTCGGGCATTGTCTTCGCTCCTTCCGGTGGGCGCGGGGCATGA
CTATCGTCGCCGCACTTATGACTGTGTTCTTTATCAT
SEQ ID NO: 6
>727 sigma70-mcjB
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGATCCGTTACTGCTTAACCAGTTATAGAGAGGATCTTGTTATCCTGGATATAAT
TAATGATAGTTTCAGCATAGTGCCTGACGCAGGTAGCTTGCTAAAAGAAAGAGATAAATTGCTTAAAGAATTCCCACAAC
TATCTTACTTTTTTGACAGTGAATATCATATTGGAAGTGTTTCTCGTAATAGTGACACTTCTTTTCTTGAAGAACGCTGG
TTTCTACCAGAACCTGACAAAACATTATATAAGTGTTCTCTATTTAAACGATTTATATTATTACTCAAAGTCTTTTACTA
TAGCTGGAATATTGAAAAAAAAGGGATGGCATGGATTTTCATAAGTAATAAAAAAGAGAATAGGCTATACTCCTTGAATG
AAGAGCATCTTATCCGGAAAGAAATTAGTAATCTTTCCATTATCTTTCATCTTAATATTTTTAAATCTGACTGTCTTACC
TATTCATACGCACTAAAAAGAATTCTTAATTCCAGAAATATTGATGCTCATCTTGTTATTGGTGTAAGGACACAACCTTT
TTATAGCCACTCTTGGGTGGAGGTTGGGGGACAAGTTATCAATGATGCTCCCAATATGCGGGATAAATTATCTGTTATTG
CAGAGATATAGTGAACTCAAAGCCCGCCGAAAGGCGGGCTTTTCTGTCCTTGAGAGTCGGGCATTGTCTTCGCTCCTTCC
GGTGGGCGCGGGGCATGACTATCGTCGCCGCACTTATGACTGTGTTCTTTATCAT
SEQ. ID. NO: 7
>1350 MBP-att linear DNA
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGAAAATCGAAGAAGGTAAACTGGTAATCTGGATTAACGGCGATAAAGGCTATAA
CGGCCTCGCTGAAGTCGGTAAGAAATTCGAGAAAGATACCGGAATTAAAGTCACCGTTGAGCATCCGGATAAACTGGAAG
AGAAATTCCCACAGGTTGCGGCAACTGGCGATGGCCCTGACATTATCTTCTGGGCACACGACCGCTTTGGTGGCTACGCT
CAATCTGGCCTGTTGGCTGAAATCACCCCGGACAAAGCGTTCCAGGACAAGCTGTATCCGTTTACCTGGGATGCCGTACG
TTACAACGGCAAGCTGATTGCTTACCCGATCGCTGTTGAAGCGTTATCGCTGATTTATAACAAAGATCTGCTGCCGAACC
CGCCAAAAACCTGGGAAGAGATCCCGGCGCTGGATAAAGAACTGAAAGCGAAAGGTAAGAGCGCGCTGATGTTCAACCTG
CLAGAACCGTACTTCACCTGGCCGCTGATTGCTGCTGACGGGGGTTATGCGTTCAAGTATGAAAACGGCAAGTACGACAT
TAAAGACGTGGGCGTGGATAACGCTGGCGCGAAAGCGGGTCTGACCTTCCTGGTTGACCTGATTAAAAACAAACACATGA
ATGCAGACACCGATTACTCCATCGCAGAAGCTGCCTTTAATAAAGGCGAAACAGCGATGACCATCAACGGCCCGTGGGCA
TGGTCCAACATCGACACCAGCAAAGTGAATTATGGTGTAACGGTACTGCCGACCTTCAAGGGTCAACCATCCAAACCGTT
CGTTGGCGTGCTGAGCGCAGGTATTAACGCCGCCAGTCCGAACAAAGAGCTGGCAAAAGAGTTCCTCGAAAACTATCTGC
TGACTGATGAAGGTCTGGAAGCGGTTAATAAAGACAAACCGCTGGGTGCCGTAGCGCTGAAGTCTTACGAGGAAGAGTTG
GCGAAAGATCCACGTATTGCCGCCACCATGGAAAACGCCCAGAAAGGTGAAATCATGCCGAACATCCCGCAGATGTCCGC
TTTCTGGTATGCCGTGCGTACTGCGGTGATCAACGCCGCCAGCGGTCGTCAGACTGTCGATGAAGCCCTGAAAGACGCGC
AGACTCGTATCACCAAGGGTGGATCTGGATGTTGTCCTGGCTGTTGCTGATGAATAACTGAATAGGGGATCCCGACTGGC
GAGAGCCAGGTAACGAATGGATCCCCGAGCTCGAGCAAAGCCCGCCGAAAGGCGGGCTTTTCTGTCCTTGAGAGTCGGGC
ATTGTCTTCGCTCCTTCCGGTGGGCGCGGGGCATGACTATCGTCGCCGCACTTATGACTGTGTTCTTTATCAT
SEQ ID NO: 8
>695 sigma70-GFP plasmid
GCATTGCTGTTCTGAGCTAACACCGTGCGTGTTGACAATTTTACCTCTGGCGGTGATAATGGTTGCAGCAAGCAATAATT
TTGTTTAACTTTAAGAAGGAGATATACAATGCGTAAAGGCGAAGAGCTGTTCACTGGTGTCGTCCCTATTCTGGTGGAAC
TGGATGGTGATGTCAACGGTCATAAGTTTTCCGTGCGTGGCGAGGGTGAAGGTGACGCAACTAATGGTAAACTGACGCTG
AAGTTCATCTGTACTACTGGTAAACTGCCGGTACCTTGGCCGACTCTGGTAACGACGCTGACTTATGGTGTTCAGTGCTT
TGCTCGTTATCCGGACCATATGAAGCAGCATGACTTCTTCAAGTCCGCCATGCCGGAAGGCTATGTGCAGGAACGCACGA
TTTCCTTTAAGGATGACGGCACGTACAAAACGCGTGCGGAAGTGAAATTTGAAGGCGATACCCTGGTAAACCGCATTGAG
CTGAAAGGCATTGACTTTAAAGAAGATGGCAATATCCTGGGCCATAAGCTGGAATACAATTTTAACAGCCACAATGTTTA
CATCACCGCCGATAAACAAAAAAATGGCATTAAAGCGAATTTTAAAATTCGCCACAACGTGGAGGATGGCAGCGTGCAGC
TGGCTGATCACTACCAGCAAAACACTCCAATCGGTGATGGTCCTGTTCTGCTGCCAGACAATCACTATCTGAGCACGCAA
AGCGTTCTGTCTAAAGATCCGAACGAGAAACGCGATCATATGGTTCTGCTGGAGTTCGTAACCGCAGCGGGCATCACGCA
TGGTATGGATGAACTGTACAAATGATGAACTCAAAGCCCGCCGAAAGGCGGGCTTTTCTGTCCTTGAGAGTCGGGCATTG
TCTTCGCTCCTTCCGGTGGGCGCGGGGCATGACTATCGTCGCCGCACTTATGACTGTGTTCTTTATCATGCAACTCGTAG
GACAGGTGCCGGCAGCGCTCTTCCGCTTCCTCGCTCACTGACTCGCTGCGCTCGGTCGTTCGGCTGCGGCGAGCGGTATC
AGCTCACTCAAAGGCGGTAATACGGTTATCCACAGAATCAGGGGATAACGCAGGAAAGAACATGTGAGCAAAAGGCCAGC
AAAAGGCCAGGAACCGTAAAAAGGCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTTTCCCCCTGGAAGCTCCCTCGTGCG
CTCTCCTGTTCCGACCCTGCCGCTTACCGGATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGCTGTGTGCACGAACCCCCCGTTCAGCCCGAC
CGCTGCGCCTTATCCGGTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAAAGAGTTGGTAGCTCTTGATCCGGCAAACAA
ACCACCGCTGGTAGCGGTGGTTTTTTTGTTTGCAAGCAGCAGATTACGCGCAGAAAAAAAGGATCTCAAGAAGATCCTTT
GATCTTTTCTACGGGGTCTGACGCTCAGTGGAACGAAAACTCACGTTAAGGGATTTTGGTCATGAGATTATCAAAAAGGA
TCTTCACCTAGATCCTTTTAAATTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCATAGTTGCCTGACTCCCCGTCGT
GTAGATAACTACGATACGGGAGGGCTTACCATCTGGCCCCAGTGCTGCAATGATACCGCGTGACCCACGCTCACCGGCTC
CAGATTTATCAGCAATAAACCAGCCAGCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGCGCAACGTTGTTGCCATTGCTACAGG
CATCGTGGTGTCACGCTCGTCGTTTGGTATGGCTTCATTCAGCTCCGGTTCCCAACGATCAAGGCGAGTTGCATGATCCC
CCATGTTGTGCAAAAAAGCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTGACTGGTGAGTACTCAAC
CAAGTCATTCTGAGAATAGTGTATGCGGCGACCGAGTTGCTCTTGCCCGGCGTCAACACGGGATAATACCGCGCCACATA
GCAGAACTTTAAAAGTGCTCATCATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGAATACTCATACTCTTCCTTTTTCAATATT
ATTGAAGCATTTATCAGGGTTATTGTCTCATGAGCGGATACATATTTGAATGTATTTAGAAAAATAAACAAATAGGGGTT
CCGCGCACATTTCCCCGAAAAGTGCCACCTGACGTCTAAGAAACCATTATTATCATGACATTAACCTATAAAAATAGGCG
TATCACGAGGCCCTTTCGTGTTCAAGAATTCTGGCGAATCCTCTGACCAGCCAGAAAACGACCTTTCTGTGGTGAAACCG
GATGCTGCAATTCAGAGCGCCAGCAAGTGGGGGACAGCAGATGACCTGACCGCCGCAGAGTGGATGTTTGACATGGTGAT
GACTATCGCACCATCAGCCAGAAAACCGAATTTTGCTGGGTGGGCTAACGATATCCGCCTGATGCGTGAACGTGACGGAC
GTAACGAAGACAACCAC
SEQ ID NO: 9
>688 T7-P1 promoter region
GCATTGCTGTTCTAATACGACTCACTATAGGGAAGC
SEQ ID NO: 10
>696 T7-P2 promoter region
GCATTGCTGTTCAGATCTCGATCCCGCGAAATTAATACGACTCACTATAGGGAGACCACAACGGTTTCCCTCTAGAAAGC
SEQ ID NO: 11
>697 T7-P3 promoter region
GCATTGCTGTTCAGATCTCGATCCCGCGAAATTAATACGACTCACTATAGGGGAATTGTGAGCGGATAACAATTCCCCTC
TAGAAAGC
SEQ ID NO: 12
>698 T7-P4 promoter region
GCATTGCTGTTCAGATCTCGATCCCGCGAAATTAATACGACTCACTATAGGGAGACGACAACGGTTTCCCTCTAGAAAGC
SEQ ID NO: 13
>699 T7-P5 promoter region
GCATTGCTGTTCAGATCTCGATCCCGCGAAATTAATACGACTCACTATAGGGAGACAACAACGGTTTCCCTCTAGAAAGC
SEQ ID NO: 14
>30810f
CAACCACGCATTGCTGTT
SEQ ID NO: 15
>30810r
CAATGCCCGACTCTCAAG
SEQ ID NO: 16
>sigma70-CDS1
AAAACCGAATTTTGCTGGGTGGGCTAACGATATCCGCCTGATGCGTGAACGTGACGGACGTAACGAAGACAACCACGCAT
TGCTGTTCTGAGCTAACACCGTGCGTGTTGACAATTTTACCTCTGGCGGTGATAATGGTTGCAGCAAGCAATAATTTTGT
TTAACTTTAAGAAGGAGATATACAATGTGCATGGACCGAATTGAAAAACTGATCAAAAAAGTCTCCAAACCAGCCCGACT
GTCCGTTGAACGATGCCGCCTGTATACAGAGAGCATGAAACAGACGGAAGGTGAACCCATGATCATTCGTCAGGCAAAAG
CCTTAAAACATGTTCTGGAAAACATTCCTATCCAGATCCTGGATTCGGAATTGATAGTGGGGACTATGCTGCCGAATCCT
CCTGGGGCGATTATCTTCCCGGAGGGGGTTGGCCTGCGCATCATTAACGAGCTCGACAGCTTACCGAATCGGGAAACTAA
TCGCCTCATGGTTGATGAAGAGGATGCCAAAGTGCTGCGTGAAGAAATTGCTCCGTATTGGCAGCGTAAAACCATCGAAG
CGTTTGCTTTTCCACTTATGCCCGACATCATGCAAATATTATATACCGGCTCAGTATTCGTTTTAACGGAGATTGCGGGT
ATTTCACATGTTGCAGTTAATTATCCGTACCTGCTGAGAAGAGGTTTCCGCTGGTTTTTGGAAGAATCGGAACGCCGTAT
ACGCGCCCTGGAGGAAAGTGGCGTTTATGAAGGTGAAAAATACTCTTTCTATCAGGCGGCAAAAATTGTGAGTGAAGCCG
TGATTAACTACGGTTTGCGTTATTCGAAACTGGCGGAGGAGCTGGCCGAAAGCGAAGATGGCGAAAGAAGGGAAGAACTG
CTAAAAATCGCAGAAATCTGTCGCAAAGTGCCGGCGGAAAAGCCAGAAACCTTCTGGGAAGCAGTGCAGTTTGTGTGGTT
GGTCCAGTCAGCCCTCCACCAAGAAAACTATGAACAGGCGATCAGCATGGGCCGGATTGACCAATATCTTTATCCGTTTT
TTAAGAAAGATATTGGTGAGGGACGCATCAATCGTGAACTGGCCTTTGACATCCTGGCTAATCTATGGATCAAAACAAAT
GAAATCGTTCCGGCTTTCGACAGCCTACTCGAGCAGTACTTCAGCGGCCAGGCGACAAATCAGGCAGTGACTATTGGTGG
TTGTGATATCTACGGCAATGATGCAACCAACGAGCTGACATATCTGATGCTGGAAGTGACGGATCGCCTGCGACTACGTC
AACCGAACGTCCATGTCCGTATTAATAAGGGATCCCCTGAGAGCTTTCTGAAGCGCCTTGCAGAAGCGATTTCTTCGGGT
TGTAACAATCTGGCGTTATTTTTTGACGATGCGGCTGTCAAAGCTTTAAAAAACGCAGAAGTAGATGATCGCGACGCTCT
GAACTACACGACCGATGGGTGTGTCGAGATTGCCCCGTTTGGTAACAGTTTTACTTCTTCCGATGCGGCACTTATCAATG
TGGCGAAAGCCTTGGAATATGCACTGAATGAAGGTGTGGATCTGCAGTTCGGCTATGAATTTGGGGCCAAGACCGAAAAG
CCAAAATTTCTAGAGGACCTGTTGGAGAAACTTCGGGAGCAAGTATCTCACATTGTGAAACTCGTAGTGCGCGGCAGCAA
CGTACTCTCTTACGCAAACGCTGAGGTAAAACCGACCCCTTTGTTGAGCTTATGCGTCGAGGACTGTTTCGAAAAGGGTG
TCGATGTGTCACGCGGTGGTGCGCGTTACAACTTTACGGGGATACAGGCGGTGGGCATTGCTGATGTAGGTGACTCCCTG
GTTGCCATAGAAGGCGCTCTGAACGCTGGTTACTCTATGGACGACATTGTTGAGGCGTGCCGCAAAAATTTCGTTGGCTA
TGAAAAACTGCACAAATTGTTGTTACAATCTCCGAAATACGGCAATGATGATGATGCTGCGGATAAGTACACAAAAATGG
TATTAGAATGGTACTGCGAAGAAGTTAACCGCCATCGTAACTTCAGGGGGGGAAAATTCGCAGCCGGCTGTTACCCTATG
ACGACGAACGTAGGATTCGGTTTTTTCACCAGCGCGCTGCCATCGGGTCGTAAATCAGGCGAACCACTGAACCCAGGCGT
GTCCCCCTCAACCGGAATGGATAGGGAGGGCGTCACCGCAGTCATTAACAGTGCCAGCAAGCTGTCGTATGAGAATCTCC
CGAACGGTGCATCTTTGACTATTAATCTATCCAGTGATGTACTTGGAGAGAAGGGAGATGCGGTGATTGAAGCGCTGATC
AAATCAAGTATGGAATTAGGCGTGATGCATGTGCAGTTTAATATCCTTAAAGAGGACCTGCTTCGTAAGGCGCAGCAAGA
ACCGGAGAAATATCGTTGGCTGTTAGTTCGCGTTGCCGGGTGGAGTGCCTATTTTGTTGAACTGAGCCGTCCGGTACAAG
AAGAGGTGATTCGTCGGATAAGCTGCCGCATCTGAATAACTGAATAGGGGATCCCGACTGGCGAGAGCCAGGTAACGAAT
GGATCCCCGAGCTCGAGCAAAGCCCGCCGAAAGGCGGGCTTTTCTGTCCTTGAGAGTCGGGCATTGTCTTCGCTCCTTCC
GGTGGGCGCGGGGCATGACTATCGTCGCCGCACTTATGACTGTGTTCTTTATCAT
SEQ ID NO: 17
>T7-CDS1
AAAACCGAATTTTGCTGGGTGGGCTAACGATATCCGCCTGATGCGTGAACGTGACGGACGTAACGAAGACAACCACGCAT
TGCTGTTCAGATCTCGATCCCGCGAAATTAATACGACTCACTATAGGGAGACGACAACGGTTTCCCTCTAGAAAGCAATA
ATTTTGTTTAACTTTAAGAAGGAGATATACAATGTGCATGGACCGAATTGAAAAACTGATCAAAAAAGTCTCCAAACCAG
CCCGACTGTCCGTTGAACGATGCCGCCTGTATACAGAGAGCATGAAACAGACGGAAGGTGAACCCATGATCATTCGTCAG
GCAAAAGCCTTAAAACATGTTCTGGAAAACATTCCTATCCAGATCCTGGATTCGGAATTGATAGTGGGGACTATGCTGCC
GAATCCTCCTGGGGCGATTATCTTCCCGGAGGGGGTTGGCCTGCGCATCATTAACGAGCTCGACAGCTTACCGAATCGGG
AAACTAATCGCCTCATGGTTGATGAAGAGGATGCCAAAGTGCTGCGTGAAGAAATTGCTCCGTATTGGCAGCGTAAAACC
ATCGAAGCGTTTGCTTTTCCACTTATGCCCGACATCATGCAAATATTATATACCGGCTCAGTATTCGTTTTAACGGAGAT
TGCGGGTATTTCACATGTTGCAGTTAATTATCCGTACCTGCTGAGAAGAGGTTTCCGCTGGTTTTTGGAAGAATCGGAAC
GCCGTATACGCGCCCTGGAGGAAAGTGGCGTTTATGAAGGTGAAAAATACTCTTTCTATCAGGCGGCAAAAATTGTGAGT
GAAGCCGTGATTAACTACGGTTTGCGTTATTCGAAACTGGCGGAGGAGCTGGCCGAAAGCGAAGATGGCGAAAGAAGGGA
AGAACTGCTAAAAATCGCAGAAATCTGTCGCAAAGTGCCGGCGGAAAAGCCAGAAACCTTCTGGGAAGCAGTGCAGTTTG
TGTGGTTGGTCCAGTCAGCCCTCCACCAAGAAAACTATGAACAGGCGATCAGCATGGGCCGGATTGACCAATATCTTTAT
CCGTTTTTTAAGAAAGATATTGGTGAGGGACGCATCAATCGTGAACTGGCCTTTGACATCCTGGCTAATCTATGGATCAA
AACAAATGAAATCGTTCCGGCTTTCGACAGCCTACTCGAGCAGTACTTCAGCGGCCAGGCGACAAATCAGGCAGTGACTA
TTGGTGGTTGTGATATCTACGGCAATGATGCAACCAACGAGCTGACATATCTGATGCTGGAAGTGACGGATCGCCTGCGA
CTACGTCAACCGAACGTCCATGTCCGTATTAATAAGGGATCCCCTGAGAGCTTTCTGAAGCGCCTTGCAGAAGCGATTTC
TTCGGGTTGTAACAATCTGGCGTTATTTTTTGACGATGCGGCTGTCAAAGCTTTAAAAAACGCAGAAGTAGATGATCGCG
ACGCTCTGAACTACACGACCGATGGGTGTGTCGAGATTGCCCCGTTTGGTAACAGTTTTACTTCTTCCGATGCGGCACTT
ATCAATGTGGCGAAAGCCTTGGAATATGCACTGAATGAAGGTGTGGATCTGCAGTTCGGCTATGAATTTGGGGCCAAGAC
CGAAAAGCCAAAATTTCTAGAGGACCTGTTGGAGAAACTTCGGGAGCAAGTATCTCACATTGTGAAACTCGTAGTGCGCG
GCAGCAACGTACTCTCTTACGCAAACGCTGAGGTAAAACCGACCCCTTTGTTGAGCTTATGCGTCGAGGACTGTTTCGAA
AAGGGTGTCGATGTGTCACGCGGTGGTGCGCGTTACAACTTTACGGGGATACAGGCGGTGGGCATTGCTGATGTAGGTGA
CTCCCTGGTTGCCATAGAAGGCGCTCTGAACGCTGGTTACTCTATGGACGACATTGTTGAGGCGTGCCGCAAAAATTTCG
TTGGCTATGAAAAACTGCACAAATTGTTGTTACAATCTCCGAAATACGGCAATGATGATGATGCTGCGGATAAGTACACA
AAAATGGTATTAGAATGGTACTGCGAAGAAGTTAACCGCCATCGTAACTTCAGGGGCGGAAAATTCGCAGCCGGCTGTTA
CCCTATGACGACGAACGTAGGATTCGGTTTTTTCACCAGCGCGCTGCCATCGGGTCGTAAATCAGGCGAACCACTGAACC
CAGGCGTGTCCCCCTCAACCGGAATGGATAGGGAGGGCGTCACCGCAGTCATTAACAGTGCCAGCAAGCTGTCGTATGAG
AATCTCCCGAACGGTGCATCTTTGACTATTAATCTATCCAGTGATGTACTTGGAGAGAAGGGAGATGCGGTGATTGAAGC
GCTGATCAAATCAAGTATGGAATTAGGCGTGATGCATGTGCAGTTTAATATCCTTAAAGAGGACCTGCTTCGTAAGGCGC
AGCAAGAACCGGAGAAATATCGTTGGCTGTTAGTTCGCGTTGCCGGGTGGAGTGCCTATTTTGTTGAACTGAGCCGTCCG
GTACAAGAAGAGGTGATTCGTCGGATAAGCTGCCGCATCTGAATAACTGAATAGGGGATCCCGACTGGCGAGAGCCAGGT
AACGAATGGATCCCCGAGCTCGAGCAAAGCCCGCCGAAAGGCGGGCTTTTCTGTCCTTGAGAGTCGGGCATTGTCTTCGC
TCCTTCCGGTGGGCGCGGGGCATGACTATCGTCGCCGCACTTATGACTGTGTTCTTTATCAT
SEQ ID NO: 18
>1204 T7-klebB (linear shown, plasmid used)
AAAACCGAATTTTGCTGGGTGGGCTAACGATATCCGCCTGATGCGTGAACGTGACGGACGTAACGAAGACAACCACGCAT
TGCTGTTCAGATCTCGATCCCGCGAAATTAATACGACTCACTATAGGGAGACGACAACGGTTTCCCTCTAGAAAGCAATA
ATTTTGTTTAACTTTAAGAAGGAGATATACAATGTTCAACATGACACACTACCTGCGTTTCTCCATTTACAAGAACGACC
TGGTGATCATTGATATAAATAACGACGAATATTTCATAATGAACGATGTGAATCACGAGAATATTAGCTTGCTGACTGAT
GTGGAGGAGGAACTTTTAGCCGCCGGACTTATTCAGTCACTCACCCCAATTGGAGGCGATAATGAGAATTTTTACGATGA
ACGCTGGCTCCCGAGGAAGGCAATCTTACGCAAGATTAATCCTGTGCTTTTACTGATGGTTTATAGCATCTTTGTAAAGT
GTAAAAAAAACCTGGATTCTAATGGCTATTATGGTGTCATTTACAGTCTGCAGAACGTTAAAAAAAACCACCGATGGGAT
AAATATTCGCCCGGTGACATTATCAATTGCTTAAACTTTATTATGCCGTTTAAACATTGCGAAAATCCTTGCCTAATCTA
TTCATATGCACTGGTTACCATGCTGAAAAAAGCTACGGGGAAAGGTACGCTGGTGGTTGGTGTTCGCACTCGTCCATTCA
TCAGTCATGCGTGGGTAGAACTCGACGGGGAAATCATCTCCGATAACATTTATTTGCGTGACAAACTGTCGGTAATCATG
GAAGTGTGATGAATAACTGAATAGGGGATCCCGACTGGCGAGAGCCAGGTAACGAATGGATCCCCGAGCTCGAGCAAAGC
CCGCCGAAAGGCGGGCTTTTCTGTCCTTGAGAGTCGGGCATTGTCTTCGCTCCTTCCGGTGGGCGCGGGGCATGACTATC
GTCGCCGCACTTATGACTGTGTTCTTTATCAT
SEQ ID NO: 19
>1205-T7-klebC (linear shown, plasmid used)
AAAACCGAATTTTGCTGGGTGGGCTAACGATATCCGCCTGATGCGTGAACGTGACGGACGTAACGAAGACAACCACGCAT
TGCTGTTCAGATCTCGATCCCGCGAAATTAATACGACTCACTATAGGGAGACGACAACGGTTTCCCTCTAGAAAGCAATA
ATTTTGTTTAACTTTAAGAAGGAGATATACAATGTTAATTATCACTGGCAATAAAAAAAGCACGGGCGCGGATAACTATA
TTTGCAAGCATAATATGTACATTTATGCGGATGAGAACTATGACACCTGGGATTACAAAGATACATACATAATCTTTAAA
GGCTATTGTTTTGATGAGGACGGTAACGGCATTGCCATCAATAAAGATAGTTTTCTCCCGGAAGTCCTGGATCGCTTGCC
CGAATTTAGTGGATTTTTCGTGCTCATCACAATCTCCAAAGACAAAACCGTTATATACAAGAGCTTAAGTCGCAACACTG
ATGTCTTTTACAGCGTTGATGACAATAACTTGACCATCTCGGATAATATCAAACTGCTGAGTGAACTGCTCGGCAAAAAA
ACTATTGATCCTAAATTTTTTAGTAGCTTTGTTAATAATGCATGGGTCGCGGTGTTCCTGTCGCCGATTTCTGGAATTGA
AAAAATTAATGGTGGTTGCAAATATGTTTTTGACTCTGCTGGGGTGCACGTGTTTAAACACGCTAATTTAACGCCGACCA
ATAAGGACTTCATTGAAGTGACCTCAAATATGATCAAATCGGTTTGTAAAAATAAAAAGGTGTTTTTACATCTGTCTGGC
GGGTTCGACTCCACGTTTATTTTTTATATACTAAAAAAAACGAACATCTTCTTCGAAGTTTATACCCATGCTCCTGACCG
TTACGATAATGATTCAGAAGTGAATCGTGTCCGGGAACTTTGCACTAAGAATAACGTACCGTTTCGTGTTGTGAGTGGTT
TTCCAGATATTCTCAAATCTAACAAAGAAGTGTCAATACCCTCTGACGTCAATGTGATTGAGAACGAATCCGAGGACAAC
CAGTACAACGAGCTGTTAAACAACCACGATATCGTATTCTTGAACGGCCATGGCGGGGACTGTCTATTTGTGCAAAACCC
ATCACTGAAGTCCGTACACCATCGTCTGAAACACGGACGTTTGATTAAGGGTTTGTGCAACGCTTATAAACTGTGCCGCC
TTAAGTATCTTTCTTTCACAGAGATCATCAATCCAAGAAGCCGGATTCATTGCAACAACTGGTTTAGCGACACAAAATAT
AAAGGTTTCTACCAGCATCCGCTGCTGATCAACATCGATGATTCGTCACCGGAATATGACCATATTGCCAACATGCTGTA
CTTTATGGAGTCACTGCCTCTGCAACTGAAGGGGGGAGCAATGATGTTCAGCCCATTTCTTATGAGCTGTGCATTTCGGG
TATTTATGAAATATAGGTATGACGATAATTTCTCATCCGAGCACGATCGCATTCTCGCCCGAAAAATCGCCTACAACATT
GCGCATGATATCCAACTGTTCGATGTACGTAAACGCTCGTCCAACAATCTGCTGTTCGACTTTCTGCATAAGAATAAGGA
AAAGATTCTTTTGCTGATCAACCGAGGCTTCACACAGGGTATGGGTGAGGTAACCACCGATGATCTGAAAGAATCGTTAG
AAATTAATACCAGTATTGGGATAGATGGTAATGCGACGAAATTCCTGAAACTGATGATGTTAAACCGCTATGCAGAAATG
AATATGCTTACGAAAGAGTAGTGAATAACTGAATAGGGGATCCCGACTGGCGAGAGCCAGGTAACGAATGGATCCCCGAG
CTCGAGCAAAGCCCGCCGAAAGGCGGGCTTTTCTGTCCTTGAGAGTCGGGCATTGTCTTCGCTCCTTCCGGTGGGCGCGG
GGCATGACTATCGTCGCCGCACTTATGACTGTGTTCTTTATCAT
SEQ ID NO: 20
>1381 T7-WTRNAP
CAACCACGCATTGCTGTTCTGAGCTAACACCGTGCGTGTTGACAATTTTACCTCTGGCGGTGATAATGGTTGCAGCAAGC
AATAATTTTGTTTAACTTTAAGAAGGAGATATACAATGAACACGATTAACATCGCTAAGAACGACTTCTCTGACATCGAA
CTGGCTGCTATCCCGTTCAACACTCTGGCTGACCATTACGGTGAGCGTTTAGCTCGCGAACAGTTGGCCCTTGAGCATGA
GTCTTACGAGATGGGTGAAGCACGCTTCCGCAAGATGTTTGAGCGTCAACTTAAAGCTGGTGAGGTTGCGGATAACGCTG
CCGCCAAGCCTCTCATCACTACCCTACTCCCTAAGATGATTGCACGCATCAACGACTGGTTTGAGGAAGTGAAAGCTAAG
CGCGGCAAGCGCCCGACAGCCTTCCAGTTCCTGCAAGAAATCAAGCCGGAAGCCGTAGCGTACATCACCATTAAGACCAC
TCTGGCTTGCCTAACCAGTGCTGACAATACAACCGTTCAGGCTGTAGCAAGCGCAATCGGTCGGGCCATTGAGGACGAGG
CTCGCTTCGGTCGTATCCGTGACCTTGAAGCTAAGCACTTCAAGAAAAACGTTGAGGAACAACTCAACAAGCGCGTAGGG
CACGTCTACAAGAAAGCATTTATGCAAGTTGTCGAGGCTGACATGCTCTCTAAGGGTCTACTCGGTGGCGAGGCGTGGTC
TTCGTGGCATAAGGAAGACTCTATTCATGTAGGAGTACGCTGCATCGAGATGCTCATTGAGTCAACCGGAATGGTTAGCT
TACACCGCCAAAATGCTGGCGTAGTAGGTCAAGACTCTGAGACTATCGAACTCGCACCTGAATACGCTGAGGCTATCGCA
ACCCGTGCAGGTGCGCTGGCTGGCATCTCTCCGATGTTCCAACCTTGCGTAGTTCCTCCTAAGCCGTGGACTGGCATTAC
TGGTGGTGGCTATTGGGCTAACGGTCGTCGTCCTCTGGCGCTGGTGCGTACTCACAGTAAGAAAGCACTGATGCGCTACG
AAGACGTTTACATGCCTGAGGTGTACAAAGCGATTAACATTGCGCAAAACACCGCATGGAAAATCAACAAGAAAGTCCTA
GCGGTCGCCAACGTAATCACCAAGTGGAAGCATTGTCCGGTCGAGGACATCCCTGCGATTGAGCGTGAAGAACTCCCGAT
GAAACCGGAAGACATCGACATGAATCCTGAGGCTCTCACCGCGTGGAAACGTGCTGCCGCTGCTGTGTACCGCAAGGACA
AGGCTCGCAAGTCTCGCCGTATCAGCCTTGAGTTCATGCTTGAGCAAGCCAATAAGTTTGCTAACCATAAGGCCATCTGG
TTCCCTTACAACATGGACTGGCGCGGTCGTGTTTACGCTGTGTCAATGTTCAACCCGCAAGGTAACGATATGACCAAAGG
ACTGCTTACGCTGGCGAAAGGTAAACCAATCGGTAAGGAAGGTTACTACTGGCTGAAAATCCACGGTGCAAACTGTGCGG
GTGTCGATAAGGTTCCGTTCCCTGAGCGCATCAAGTTCATTGAGGAAAACCACGAGAACATCATGGCTTGCGCTAAGTCT
CCACTGGAGAACACTTGGTGGGCTGAGCAAGATTCTCCGTTCTGCTTCCTTGCGTTCTGCTTTGAGTACGCTGGGGTACA
GCACCACGGCCTGAGCTATAACTGCTCCCTTCCGCTGGCGTTTGACGGGTCTTGCTCTGGCATCCAGCACTTCTCCGCGA
TGCTCCGAGATGAGGTAGGTGGTCGCGCGGTTAACTTGCTTCCTAGTGAAACCGTTCAGGACATCTACGGGATTGTTGCT
AAGAAAGTCAACGAGATTCTACAAGCAGACGCAATCAATGGGACCGATAACGAAGTAGTTACCGTGACCGATGAGAACAC
TGGTGAAATCTCTGAGAAAGTCAAGCTGGGCACTAAGGCACTGGCTGGTCAATGGCTGGCTTACGGTGTTACTCGCAGTG
TGACTAAGCGTTCAGTCATGACGCTGGCTTACGGGTCCAAAGAGTTCGGCTTCCGTCAACAAGTGCTGGAAGATACCATT
CAGCCAGCTATTGATTCCGGCAAGGGTCTGATGTTCACTCAGCCGAATCAGGCTGCTGGATACATGGCTAAGCTGATTTG
GGAATCTGTGAGCGTGACGGTGGTAGCTGCGGTTGAAGCAATGAACTGGCTTAAGTCTGCTGCTAAGCTGCTGGCTGCTG
AGGTCAAAGATAAGAAGACTGGAGAGATTCTTCGCAAGCGTTGCGCTGTGCATTGGGTAACTCCTGATGGTTTCCCTGTG
TGGCAGGAATACAAGAAGCCTATTCAGACGCGCTTGAACCTGATGTTCCTCGGTCAGTTCCGCTTACAGCCTACCATTAA
CACCAACAAAGATAGCGAGATTGATGCACACAAACAGGAGTCTGGTATCGCTCCTAACTTTGTACACAGCCAAGACGGTA
GCCACCTTCGTAAGACTGTAGTGTGGGCACACGAGAAGTACGGAATCGAATCTTTTGCACTGATTCACGACTCCTTCGGT
ACCATTCCGGCTGACGCTGCGAACCTGTTCAAAGCAGTGCGCGAAACTATGGTTGACACATATGAGTCTTGTGATGTACT
GGCTGATTTCTACGACCAGTTCGCTGACCAGTTGCACGAGTCTCAATTGGACAAAATGCCAGCACTTCCGGCTAAAGGTA
ACTTGAACCTCCGTGACATCTTAGAGTCGGACTTCGCGTTCGCGTAATGAATAACTGAATAGGGGATCCCGACTGGCGAG
AGCCAGGTAACGAATGGATCCCCGAGCTCGAGCAAAGCCCGCCGAAAGGCGGGCTTTTCTGTCCTTGAGAGTCGGGCATT
G
SEQ ID NO: 21
>1338 T7-MBP
AAAACCGAATTTTGCTGGGTGGGCTAACGATATCCGCCTGATGCGTGAACGTGACGGACGTAACGAAGACAACCACGCAT
TGCTGTTCAGATCTCGATCCCGCGAAATTAATACGACTCACTATAGGGAGACGACAACGGTTTCCCTCTAGAAAGCAATA
ATTTTGTTTAACTTTAAGAAGGAGATATACAATGAAAATCGAAGAAGGTAAACTGGTAATCTGGATTAACGGCGATAAAG
GCTATAACGGCCTCGCTGAAGTCGGTAAGAAATTCGAGAAAGATACCGGAATTAAAGTCACCGTTGAGCATCCGGATAAA
CTGGAAGAGAAATTCCCACAGGTTGCGGCAACTGGCGATGGCCCTGACATTATCTTCTGGGCACACGACCGCTTTGGTGG
CTACGCTCAATCTGGCCTGTTGGCTGAAATCACCCCGGACAAAGCGTTCCAGGACAAGCTGTATCCGTTTACCTGGGATG
CCGTACGTTACAACGGCAAGCTGATTGCTTACCCGATCGCTGTTGAAGCGTTATCGCTGATTTATAACAAAGATCTGCTG
CCGAACCCGCCAAAAACCTGGGAAGAGATCCCGGCGCTGGATAAAGAACTGAAAGCGAAAGGTAAGAGCGCGCTGATGTT
CAACCTGCAAGAACCGTACTTCACCTGGCCGCTGATTGCTGCTGACGGGGGTTATGCGTTCAAGTATGAAAACGGCAAGT
ACGACATTAAAGACGTGGGCGTGGATAACGCTGGCGCGAAAGCGGGTCTGACCTTCCTGGTTGACCTGATTAAAAACAAA
CACATGAATGCAGACACCGATTACTCCATCGCAGAAGCTGCCTTTAATAAAGGCGAAACAGCGATGACCATCAACGGCCC
GTGGGCATGGTCCAACATCGACACCAGCAAAGTGAATTATGGTGTAACGGTACTGCCGACCTTCAAGGGTCAACCATCCA
AACCGTTCGTTGGCGTGCTGAGCGCAGGTATTAACGCCGCCAGTCCGAACAAAGAGCTGGCAAAAGAGTTCCTCGAAAAC
TATCTGCTGACTGATGAAGGTCTGGAAGCGGTTAATAAAGACAAACCGCTGGGTGCCGTAGCGCTGAAGTCTTACGAGGA
AGAGTTGGCGAAAGATCCACGTATTGCCGCCACCATGGAAAACGCCCAGAAAGGTGAAATCATGCCGAACATCCCGCAGA
TGTCCGCTTTCTGGTATGCCGTGCGTACTGCGGTGATCAACGCCGCCAGCGGTCGTCAGACTGTCGATGAAGCCCTGAAA
GACGCGCAGACTCGTATCACCAAGGGTGGATGATGAATAACTGAATAGGGGATCCCGACTGGCGAGAGCCAGGTAACGAA
TGGATCCCCGAGCTCGAGCAAAGCCCGCCGAAAGGCGGGCTTTTCTGTCCTTGAGAGTCGGGCATTGTCTTCGCTCCTTC
CGGTGGGCGCGGGGCATGACTATCGTCGCCGCACTTATGACTGTGTTCTTTATCAT
SEQ ID NO: 22
>1339 T7-MBP-FlAsH
AAAACCGAATTTTGCTGGGTGGGCTAACGATATCCGCCTGATGCGTGAACGTGACGGACGTAACGAAGACAACCACGCAT
TGCTGTTCAGATCTCGATCCCGCGAAATTAATACGACTCACTATAGGGAGACGACAACGGTTTCCCTCTAGAAAGCAATA
ATTTTGTTTAACTTTAAGAAGGAGATATACAATGAAAATCGAAGAAGGTAAACTGGTAATCTGGATTAACGGCGATAAAG
GCTATAACGGCCTCGCTGAAGTCGGTAAGAAATTCGAGAAAGATACCGGAATTAAAGTCACCGTTGAGCATCCGGATAAA
CTGGAAGAGAAATTCCCACAGGTTGCGGCAACTGGCGATGGCCCTGACATTATCTTCTGGGCACACGACCGCTTTGGTGG
CTACGCTCAATCTGGCCTGTTGGCTGAAATCACCCCGGACAAAGCGTTCCAGGACAAGCTGTATCCGTTTACCTGGGATG
CCGTACGTTACAACGGCAAGCTGATTGCTTACCCGATCGCTGTTGAAGCGTTATCGCTGATTTATAACAAAGATCTGCTG
CCGAACCCGCCAAAAACCTGGGAAGAGATCCCGGCGCTGGATAAAGAACTGAAAGCGAAAGGTAAGAGCGCGCTGATGTT
CAACCTGCAAGAACCGTACTTCACCTGGCCGCTGATTGCTGCTGACGGGGGTTATGCGTTCAAGTATGAAAACGGCAAGT
ACGACATTAAAGACGTGGGCGTGGATAACGCTGGCGCGAAAGCGGGTCTGACCTTCCTGGTTGACCTGATTAAAAACAAA
CACATGAATGCAGACACCGATTACTCCATCGCAGAAGCTGCCTTTAATAAAGGCGAAACAGCGATGACCATCAACGGCCC
GTGGGCATGGTCCAACATCGACACCAGCAAAGTGAATTATGGTGTAACGGTACTGCCGACCTTCAAGGGTCAACCATCCA
AACCGTTCGTTGGCGTGCTGAGCGCAGGTATTAACGCCGCCAGTCCGAACAAAGAGCTGGCAAAAGAGTTCCTCGAAAAC
TATCTGCTGACTGATGAAGGTCTGGAAGCGGTTAATAAAGACAAACCGCTGGGTGCCGTAGCGCTGAAGTCTTACGAGGA
AGAGTTGGCGAAAGATCCACGTATTGCCGCCACCATGGAAAACGCCCAGAAAGGTGAAATCATGCCGAACATCCCGCAGA
TGTCCGCTTTCTGGTATGCCGTGCGTACTGCGGTGATCAACGCCGCCAGCGGTCGTCAGACTGTCGATGAAGCCCTGAAA
GACGCGCAGACTCGTATCACCAAGGGTGGATCTGGATGTTGTCCTGGCTGTTGCTGATGAATAACTGAATAGGGGATCCC
GACTGGCGAGAGCCAGGTAACGAATGGATCCCCGAGCTCGAGCAAAGCCCGCCGAAAGGCGGGCTTTTCTGTCCTTGAGA
GTCGGGCATTGTCTTCGCTCCTTCCGGTGGGCGCGGGGCATGACTATCGTCGCCGCACTTATGACTGTGTTCTTTATCAT

1. A composition for in vitro gene expression, comprising:
a treated cell lysate derived from one or more host cells such as bacteria, archaea, plant or animal;
a plurality of supplements for gene transcription and translation;
an energy recycling system for providing and recycling adenosine triphosphate (ATP); and
one or more exogenous additives selected from the group consisting of polar aprotic solvents, quaternary ammonium salts, sulfones, ectoines, glycols, amides, amines, sugar polymers, sugar alcohols, slow elongation-rate RNA polymerase (RNAP) and ribosomes, wherein preferably the sugar polymers and sugar alcohols are not for providing energy source.

2. The composition of claim 1, for use in expressing a metagenomically derived gene, a plurality of genes that together constitute a pathway, and/or synthetic proteins, wherein preferably the pathway is designed for synthesis of a natural product.

3. The composition of claim 2, wherein the gene or pathway has not been optimized for in vitro gene expression.

4. The composition of claim 1, wherein the plurality of supplements comprise magnesium and potassium salts, ribonucleotides, amino acids, a starting energy substrate, and a pH buffer.

5. The composition of claim 1, wherein the one or more additives modulate nucleic acid secondary structure, improve RNAP processivity and/or stability, affect RNAP elongation rate, improve ribosome synergy with RNAP and/or stability, and/or improve stability of polypeptide being synthesized.

6. The composition of claim 1, wherein the slow elongation-rate RNAP is homologous to the host cells, such as RNA PolI, RNA PolII, RNA PolIII, and bacterial RNAP.

7. The composition of claim 1, wherein the slow elongation-rate RNAP is heterologous to the host cells, such as SP6 RNAP variants, T7 RNAP variants, and T3 RNAP variants.

8. The composition of claim 7, wherein the slow elongation-rate RNAP is sourced from a thermophile or psychrophile.

9. The composition of claim 1, wherein the slow elongation-rate RNAP is a synthetic RNAP such as engineered T7 RNAP variants and engineered RNA PolII variants.

10. The composition of claim 9, wherein the slow elongation-rate RNAP is engineered by directed evolution and/or rational design.

11. The composition of claim 1, wherein the slow elongation-rate RNAP is provided as a purified protein or as a nucleic acid encoding the slow elongation-rate RNAP.

12. The composition of claim 1, further comprising exogenous nucleic acids to be expressed in the composition, wherein each exogenous nucleic acid comprises a promoter that is recognized by the slow elongation-rate RNAP.

13. The composition of claim 1, wherein the ribosomes are sourced from the host cells, or from an organism different than the host cells, wherein preferably the ribosomes are provided at 0.1 μM to 100 μM concentration.

14. The composition of any one of claims 1-13, wherein the composition comprises both slow elongation-rate RNAP and exogenous ribosomes, wherein preferably the slow elongation-rate RNAP and the exogenous ribosomes are coupled, wherein optionally such coupling is orthogonal to the host cells.

15. A method of preparing the composition of any one of claims 1-14, comprising:
providing an in vitro transcription/translation system comprising the treated cell lysate, the plurality of supplements and the energy recycling system; and
supplying the one or more exogenous additives.

16. A method of in vitro gene expression, comprising:
providing the composition of any one of claims 1-14, and
providing one or more nucleic acids to be expressed.
